(19) United States
(12) Patent Application Publication
(10) Pub. No.: US 2025/0052757 A1
(43) Pub. Date: Feb. 13, 2025

(54) Use of PD-L1 Low Expression Status to Select Subjects for Cancer Immunotherapy

(71) Applicants: Vaccinex, Inc., Rochester, NY (US); MSD International GmbH, Luzern (CH)

(72) Inventors: Elizabeth EVANS, Bloomfield, NY (US); Maurice ZAUDERER, Pittsford, NY (US); Terry FISHER, Rochester, NY (US)

(21) Appl. No.: 18/799,733

(22) Filed: Aug. 9, 2024

Related U.S. Application Data
(60) Provisional application No. 63/531,909, filed on Aug. 10, 2023.

(51) Int. Cl. G01N 33/574; A61K 31/675; A61K 39/00; A61K 45/06; A61P 35/00; C07K 16/28; G01N 33/68

(52) U.S. Cl. CPC G01N 33/57492; A61K 31/675; A61K 45/06; A61P 35/00; C07K 16/2818; C07K 16/2827; G01N 33/6893; A61K 2039/507; A61K 2039/545; C07K 2317/565; C07K 2317/76; G01N 2333/70596; G01N 2474/20

(57) ABSTRACT

The disclosure relates to methods for treating cancer or selecting subjects for cancer treatment using low PD-L1 expression as a patient biomarker prior to treatment.

### CROSS-REFERENCE TO RELATED APPLICATION

This application claims benefit of the filing date of U.S. Appl. No. 63/531,909, filed Aug. 10, 2023, the contents of which are incorporated by reference in their entirety.

### REFERENCE TO SEQUENCE LISTING SUBMITTED ELECTRONICALLY

The content of the electronically submitted sequence listing in ST26 file (Name: 5895-0001US01 SEQLXML.xml; Size: 17,209 bytes; and Date of Creation: Aug. 8, 2024) filed with the application is incorporated herein by reference in its entirety.

### FIELD OF THE DISCLOSURE

The disclosure relates to methods for treating cancer or selecting subjects for combination cancer immunotherapy treatment using low PD-L1 expression as a patient biomarker.

### BACKGROUND

Semaphorin 4D (SEMA4D), also known as CD100, is a transmembrane protein that belongs to the semaphorin gene family. SEMA4D is expressed on the cell surface as a homodimer, but upon cell activation SEMA4D can be released from the cell surface via proteolytic cleavage to generate sSEMA4D, a soluble form of the protein, which is also biologically active. See Suzuki et al., Nature Rev. Immunol. 3:159-167 (2003); Kikutani et al., Nature Immunol. 9:17-23 (2008)

SEMA4D is expressed at high levels in lymphoid organs, including the spleen, thymus, and lymph nodes, and in non-lymphoid organs, such as the brain, heart, and kidney. In lymphoid organs, SEMA4D is abundantly expressed on resting T cells but only weakly expressed on resting B cells and antigen-presenting cells (APCs), such as dendritic cells (DCs). Its expression, however, is upregulated in these cells following activation by various immunological stimuli. The release of soluble SEMA4D from immune cells is also increased by cell activation. SEMA4D has been implicated in the development of certain cancers (Ch'ng et al., Cancer 110:164-72 (2007); Campos et al., Oncology Letters, 5:1527-35 (2013); Kato et al., Cancer Sci. 102:2029-37 (2011)) and several reports suggest that one mechanism of this influence is the role of SEMA4D in promoting tumor angiogenesis (Conrotto et al., Blood 105:4321-4329 (2005). Basile et al., J Biol. Chem. 282: 34888-34895 (2007); Sierra et. al. J. Exp. Med. 205:1673 (2008); Zhou et al., Angiogenesis 15:391-407 (2012)). Tumor growth and metastasis involve a complex process of cross talk amongst the tumor cells, stroma and immune infiltrate, as well as the endothelial cells and vasculature. SEMA4D is over-expressed in a wide array of tumor types and is also produced by inflammatory cells recruited to the tumor microenvironment.

Immunosuppressive myeloid cells in the tumor microenvironment (TME) limit the efficacy of immune checkpoint inhibitors (ICI) in cancer. Preclinical and clinical studies demonstrated that antibody blockade of SEMA4D in combination with ICI promotes infiltration of CD8+ cytotoxic T cells and inhibits recruitment and function of myeloid derived suppressor cells (MDSC) in tumors leading to enhanced efficacy of ICI (Clavijo et al., Cancer Immunol Res. 7:282-291 (2019)).

It has now been found from an interim analysis of results for combination immunotherapy with pepinemab and pembrolizumab in the KEYNOTE-B84 study (NCT04815720), that the Objective Response Rate (ORR) and Progression Free Survival (PFS) for a PD-L1 low population (combined positive score (CPS)<20) is almost 2× that of the historical control for this population reported in the KEYNOTE-048 study for single agent pembrolizumab, while responses in the CPS ≥20 population to combination therapy were similar to single agent pembrolizumab, presenting a new and unexpected biomarker for selecting patients who will benefit from such combination therapies.

Hence the present disclosure addresses a need in the art for additional methods of defining populations of cancer patients that are likely to benefit from treatment with an anti-SEMA4D antibody or antigen binding fragment thereof, for example pepinemab, in combination with other immunotherapeutic agents, particularly ICIs.

### SUMMARY OF THE DISCLOSURE

The present disclosure relates to the use of PD-L1 status as a biomarker to select and treat cancer patients with combination immunotherapy.

In one aspect, the embodiments of the disclosure provide for a method of selecting subjects having or suspected of having cancer for treatment with an isolated antibody or antigen-binding fragment thereof that specifically binds to semaphorin-4D (SEMA4D) and an effective amount of at least one other immune modulating therapy, preferably at least one immune checkpoint inhibitor, which comprises (a) determining PD-L1 expression levels in tumor tissue or cancerous cells of a subject; and (b) selecting a subject for treatment if (i) the tumor tissue is identified as having a CPS of <25 for PD-L1 expression or a tumor proportion score (TPS) ranging from <50% to <80% for PD-L1 expression, or (ii) the cancerous cells are identified as having a TPS ranging from <50% to <80% for PD-L1 expression. In some of these embodiments, the CPS is <20. In some embodiments, the CPS is <10. In some of these embodiments, the TPS is <50%.

In another aspect, the embodiments of the disclosure provide for a method for treating, inhibiting, delaying, or reducing malignant cell growth in a subject with cancer, which comprises (a) determining PD-L1 expression levels in tumor tissue or cancerous cells of the subject; and (b) administering to the subject an effective amount of an isolated antibody or antigen-binding fragment thereof that specifically binds to semaphorin-4D (SEMA4D) and an effective amount of at least one other immune modulating therapy, preferably at least one immune checkpoint inhibitor, wherein the tumor tissue or cells are identified as having either or both of a CPS of <25 for PD-L1 expression or a TPS ranging from <50% to <80%, thereby treating the subject. In some of these embodiments, the CPS is <20. In some embodiments, the CPS is <10. In some of these embodiments, the TPS is <50%.

In a further aspect, the embodiments of the disclosure provide for a method of treating cancer in a human patient identified as having a tumor with low PD-L1 expression which comprises administering to the patient, as combination immunotherapy, (a) an anti-semaphorin-4D (SEMA4D) antibody, or an antigen binding fragment thereof, and (b) an immune checkpoint inhibitor, wherein the tumor has a combined positive score (CPS) of <25 before the immune checkpoint inhibitor is administered. In some of these embodiments, the CPS is <20. In some embodiments, the CPS is <10.

In a still further aspect, the embodiments of the disclosure provide for a method of treating cancer in a human patient identified as having a tumor with low PD-L1 expression which comprises administering to the patient, as combination immunotherapy, (a) an anti-semaphorin-4D (SEMA4D) antibody, or an antigen binding fragment thereof, and (b) an immune checkpoint inhibitor, wherein the tumor has a total proportion score (TPS) ranging from <50% to <80% before the immune checkpoint inhibitor is administered. In some of these embodiments, the TPS is <50%.

In some embodiments of any of the methods and uses described herein, PD-L1 expression levels are determined by immunohistochemical analysis.

In some embodiments of any of the methods and uses described herein, the anti-SEMA4D antibody or antigen-binding fragment thereof inhibits SEMA4D interaction with its receptor. In some of these embodiments, the receptor is Plexin-B1, Plexin-B2, CD72, or any combination thereof. In some of these embodiments, the anti-SEMA4D antibody or fragment thereof inhibits SEMA4D-mediated signal transduction.

In some embodiments of any of the methods and uses described herein, the anti-SEMA4D antibody or antigen-binding fragment thereof is selected from the group consisting of
(i) an antibody or antigen binding fragment thereof comprising a variable heavy chain (VH) region having VH CDRs 1-3 comprising amino acid sequences SEQ ID NOS:1, 2, and 3, respectively, and a variable light chain (VL) region comprising VL CDRs 1-3 comprising SEQ ID NOS:4, 5, and 6, respectively;
(ii) the antibody of (i), or antigen binding fragment thereof, wherein the VH and VL chains comprise, respectively, SEQ ID NO:7 and SEQ ID NO:8 (human), or SEQ ID NO:9 and SEQ ID NO:10 (mouse);
(iii) an antibody or antigen binding fragment thereof comprising a VH region having VH CDRs 1-3 comprising amino acid sequences SEQ ID NOS:11, 12, and 13, respectively; and a VL region comprising VL CDRs 1-3 comprising amino acid sequences SEQ ID NOS:14, 15, and 16, respectively; and
(iv) the antibody of (ii), or antigen binding fragment thereof, wherein the VH and VL chains comprise, respectively, SEQ ID NO:17 and SEQ ID NO:18.
   For ease of understanding, (i) provides the six CDRs for mouse/human MAbVX15/2503; (ii) provides the antigen binding domains for mouse/human MAbVX15/2503; (iii) provides the six CDRs for MAb D2517; and (iv) provides the antigen binding domain for MAb D2517.

In some embodiments of any of the methods and uses described herein, the anti-SEMA4D antibody is pepinemab or an antigen-binding fragment thereof. In some embodiments of any of the methods and uses described herein, the anti-SEMA4D antibody or an antigen-binding fragment thereof is administered at a dose of 20 mg/kg.

In some embodiments of any of the methods and uses described herein, the at least one immune checkpoint inhibitor is an anti-CTLA4 antibody, an anti-PD-1 antibody, an anti-PD-L1 antibody, an anti-LAG3 antibody, an anti-TIGIT antibody, an anti-B7-H3 antibody or a combination thereof.

In some embodiments of any of the methods and uses described herein, the anti-PD-1 antibody is pembrolizumab, cemiplimab, dostarlimab or nivolumab, and preferably is pembrolizumab. In select embodiments of any of the methods and uses described herein, when the anti-PD1 antibody is pembrolizumab, it is administered at a dose of about 200 mg.

In some embodiments of any of the methods and uses described herein, the isolated anti-SEMA4D antibody, or antigen-binding fragment thereof, and the at least one other immune modulating therapy, preferably the at least one immune checkpoint inhibitor, are administered separately or concurrently. In some of these embodiments, these two drugs are administered every three weeks, i.e., on the same treatment cycle.

In some embodiments of any of the methods and uses described herein, administration of the combination of the isolated anti-SEMA4D antibody, or antigen-binding fragment thereof, and the at least one other immune modulating therapy, preferably the at least one immune checkpoint inhibitor, results in enhanced therapeutic efficacy relative to administration of the at least one other immune modulating therapy, preferably the at least one immune checkpoint inhibitor, alone, wherein such enhancement is an approximately two-fold improvement in the objective response rate (ORR).

In some embodiments of any of the methods and uses described herein, the treatment methods further comprise administering an immune modulating therapy, radiation or chemotherapy.

In some embodiments, the immune modulating therapy is selected from the group consisting of administration of a cancer vaccine, administration of an immunostimulatory agent, adoptive T cell or antibody therapy, administration of an immune checkpoint inhibitor, administration of a regulatory T cell (Treg) modulator, and a combination thereof.

In some embodiments, the immune modulating therapy comprises a second immune checkpoint inhibitor. In some embodiments, the second immune checkpoint inhibitor is selected from the group consisting of an antibody or antigen-binding fragment thereof that specifically binds to CTLA4, PD-1, PD-L1, LAG3, TIM3, B7-H3, TIGIT or any combination thereof. In some embodiments, the antibody or antigen-binding fragment of the second immune checkpoint inhibitor comprises the anti-PD-L1 antibody avelumab, atezolizumab, durvalumab.

In some embodiments, the immune modulating therapy comprises administration of a cancer vaccine.

In some embodiments, the immune modulating therapy comprises administration of a Treg modulator. In some embodiments, the Treg modulator is cyclophosphamide

In some embodiments of any of the methods and uses described herein, the cancer is carcinoma, lymphoma, blastoma, sarcoma, leukemia, squamous cell cancer, small-cell lung cancer, non-small cell lung cancer, adenocarcinoma of the lung, squamous carcinoma of the lung, cancer of the peritoneum, hepatocellular cancer, gastrointestinal cancer, gastric cancer, pancreatic cancer, neuroendocrine cancer, glioblastoma, cervical cancer, ovarian cancer, liver cancer, bladder cancer, brain cancer, hepatoma, breast cancer, colon cancer, colorectal cancer, endometrial or uterine carcinoma, esophageal cancer, salivary gland carcinoma, kidney cancer, liver cancer, prostate cancer, vulval cancer, thyroid cancer, head and neck cancer, or any combination thereof.

In certain embodiments of any of the methods and uses described herein, the cancer is selected from the group consisting of locally advanced or recurrent or metastatic (in all of the following) head and neck cancer, non-small cell lung cancer, cervical cancer, urothelial carcinoma, esophageal squamous cell carcinoma (ESCC), renal cell carcinoma, ovarian cancer, prostate cancer, bladder cancer, pancreatic cancer, gastrointestinal cancer, hepatocellular carcinoma, sarcoma, melanoma, triple negative breast cancer (TNBC), microsatellite instability/mismatch repair (MSI/MMR) deficient tumors and colorectal cancer.

### BRIEF DESCRIPTION OF THE DRAWINGS

FIG. 1. Study results for treating R/M HNSCC patients with pepinemab and pembrolizumab combination as described in Example 1. This table shows objective responses and progression free survival (PFS) for patients with PD-L1 combined positive score (CPS)<20 (PDL1-Low) and for patients with PD-L1 CPS ≥20 (PD-L1 High) receiving the combination therapy (KEYNOTE-B84) at the interim analysis (n=36 total) compared to historical control of patients on pembrolizumab alone (KEYNOTE-048).* NOTE: KEYNOTE-048 CPS <20 was calculated post-hoc from analysis of CPS<1 and 1-19 assessments; these do not represent alpha controlled analyses. PFS shown was reported for CPS 1-19. **This table includes overall survival (OS) for patients with PD-L1 combined positive score (CPS)<20 (PDL1-Low) and for patients with PD-L1 CPS ≥20 (PD-L1 High) receiving the combination therapy (KEYNOTE-B84), including 49 patients total at the median follow up of ˜14.5 months, compared to historical control of patients on pembrolizumab alone (KEYNOTE-048). Total represents the number of patients in each arm of the study.

Abbreviations: CR, complete response; PR, partial response; SD, stable disease; ORR, objective response rate; DCR, disease control rate, sum of the complete, partial and stable disease rates; PFS, progression-free survival; KEYNOTE-B84 clinical study with pepinemab and pembrolizumab for treatment of recurrent/metastatic head and neck cancer; KEYNOTE-048 clinical study with pembrolizumab; PEPI, pepinemab; PEMBRO, pembrolizumab; R/M HNSCC, recurrent/metastatic head and neck squamous cell carcinoma; NR: not reached; CI: confidence interval.

FIG. 2. Detailed breakdown of the study results from KEYNOTE-B84 for treating R/M HNSCC with pepinemab and pembrolizumab combination for PDL-1 Low responders from FIG. 1. This table shows the objective responses and progression free survival (PFS) for patients with PD-L1 of CPS <1 and PD-L1 of CPS 1-19 to the combination therapy (KN-B84) at the interim analysis compared to patients on pembrolizumab alone (KN-048). **This table includes overall survival (OS) for patients with PD-L1 combined positive score (CPS)<20 (PDL1-Low) and for patients with PD-L1 CPS ≥20 (PD-L1 High) receiving the combination therapy (KEYNOTE-B84) at the median follow up of ˜14.5 months, compared to historical control of patients on pembrolizumab alone (KEYNOTE-048). Total represents the number of patients in each arm of the study. Abbreviations are the same as for FIG. 1.

FIG. 3. Results from individual R/M HNSCC patients treated with pepinemab and pembrolizumab combination as described in Example 1. FIG. 3 graphically illustrates the percentage change in target lesion diameter relative to the baseline scan for individual patients with PD-L1 of CPS <20 over time (weeks). Lines are coded based on best objective response; x indicates patient discontinued the study and (†) indicates patient died. Key & Abbreviations: CR, complete response (●); PR, partial response (□); SD, stable disease ( [symbol] ); PD, progressive disease ( [symbol] ).

FIG. 4. Results from individual R/M HNSCC patients treated with pepinemab and pembrolizumab combination as described in Example 1. FIG. 4 graphically illustrates the percentage change in target lesion diameter relative to the baseline scan for individual patients with PD-L1 of CPS ≥20 over time (weeks). Lines are coded based on best objective response. Key and abbreviations are the same as for FIG. 3.

FIG. 5. Progression-free survival treating R/M HNSCC patients with pepinemab and pembrolizumab combination as described in Example 1. FIG. 5 graphically illustrates the probability of progression-free survival over time (in months) for patients with PD-L1 of CPS <20 (black line) or a PD-L1 of CPS ≥20 (grey line).

FIG. 6. Overall survival treating R/M HNSCC patients with pepinemab and pembrolizumab combination as described in Example 1. FIG. 6 graphically illustrates the probability of overall survival over time (in months) for patients with PD-L1 of CPS <20 (black line), PD-L1 of CPS 1-19 (dashed black line), or a PD-L1 of CPS ≥20 (grey line).

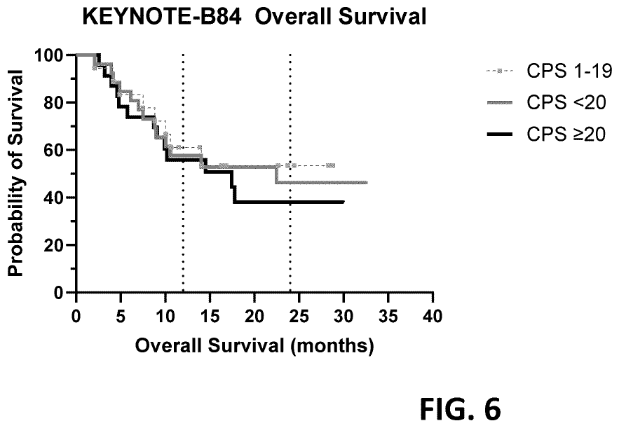

### DETAILED DESCRIPTION OF THE DISCLOSURE

### I. Definitions

In order that the present disclosure may be more readily understood, certain terms are defined below. Additional definitions may be found within the detailed description of the disclosure.

It is to be noted that the term “a” or “an” entity refers to one or more of that entity; for example, “a polynucleotide,” is understood to represent one or more polynucleotides. As such, the terms “a” (or “an”), “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or embodiments of the disclosure, which can be had by reference to the specification as a whole. Accordingly, the terms defined immediately below are more fully defined by reference to the specification in its entirety.

Wherever embodiments are described with the language “comprising,” otherwise analogous embodiments described in terms of “consisting of” and/or “consisting essentially of” are also provided.

Amino acids are referred to herein by their commonly known three letter symbols or by the one-letter symbols recommended by the IUPAC-IUB Biochemical Nomenclature Commission. Nucleotides, likewise, are referred to by their commonly accepted single-letter codes.

As used herein, the terms “cancer” and “cancerous” refer to or describe the physiological condition in mammals in which a population of cells are characterized by unregulated cell growth. Examples of cancer include, but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, gastric, pancreatic cancer, glioblastoma, cervical cancer, ovarian cancer, liver cancer, bladder cancer, brain cancer, hepatoma, breast cancer, colon cancer, colorectal cancer, endometrial or uterine carcinoma, esophageal cancer, salivary gland carcinoma, sarcoma, kidney cancer, liver cancer, prostate cancer, vulval cancer, thyroid cancer, hepatic carcinoma and various types of head and neck cancers.

In certain embodiments, metastatic cancers that are amenable to treatment via the methods provided herein include, but are not limited to metastatic sarcomas, breast carcinomas, ovarian cancer, head and neck cancer, lung cancers and pancreatic cancer. In certain embodiments metastatic cancers or tumor cells that are amenable to treatment via the methods provided herein express Plexin-B1 and/or Plexin-B2 receptors for SEMA4D.

As used herein, the term “clinical laboratory” refers to a facility for the examination or processing of materials derived from a living subject, e.g., a human being. Non-limiting examples of processing include biological, biochemical, serological, chemical, immunohematological, hematological, biophysical, cytological, pathological, genetic, or other examination of materials derived from the human body for the purpose of providing information, e.g., for the diagnosis, prevention, or treatment of any disease or impairment of, or the assessment of the health of living subjects, e.g., human beings. These examinations can also include procedures to collect or otherwise obtain a sample, prepare, determine, measure, or otherwise describe the presence or absence of various substances in the body of a living subject, e.g., a human being, or a sample obtained from the body of a living subject, e.g., a human being.

The terms “proliferative disorder” and “proliferative disease” refer to disorders associated with abnormal cell proliferation such as cancer.

“Tumor” and “neoplasm” as used herein refer to any mass of tissue that result from excessive cell growth or proliferation, either benign (noncancerous) or malignant (cancerous) including pre-cancerous lesions. In certain embodiments, tumors described herein express Plexin-B1 and/or Plexin-B2, and can express SEMA4D and activated Met.

As used herein, the term “healthcare benefits provider” encompasses individual parties, organizations, or groups providing, presenting, offering, paying for in whole or in part, or being otherwise associated with giving a patient access to one or more healthcare benefits, benefit plans, health insurance, and/or healthcare expense account programs.

The term “immune modulating therapy” or “immunotherapy” refers to treatment that impacts a disease or disorder in a subject by inducing and/or enhancing an immune response in that subject. Immune modulating therapies include cancer vaccines, immunostimulatory agents, adoptive T cell or antibody therapy, and immune checkpoint inhibitors (Lizée et al. 2013. Harnessing the Power of the Immune System to Target Cancer. Annu. Rev. Med. Vol. 64 No. 71-90).

The term “immune modulating agent” refers to the active agents of immunotherapy. Immune modulating agents include a diverse array of recombinant, synthetic and natural, preparation. Examples of immune modulating agents include, but are not limited to, interleukins such as IL-2, IL-7, IL-12; cytokines such as granulocyte colony-stimulating factor (G-CSF), interferons; various chemokines such as CXCL13, CCL26, CXCL7; antagonists of immune checkpoint blockades such as anti-CTLA-4, anti-PD1 or anti-PD-L1 (ligand of PD-1), anti-LAG3, anti-B7-H3, synthetic cytosine phosphate-guanosine (CpG) oligodeoxynucleotides, glucans; and modulators of regulatory T cells (Tregs) such as cyclophosphamide.

The terms “metastasis,” “metastases,” “metastatic,” and other grammatical equivalents as used herein refer to cancer cells which spread or transfer from the site of origin (e.g., a primary tumor) to other regions of the body with the development of a similar cancerous lesion at the new location. A “metastatic” or “metastasizing” cell is one that loses adhesive contacts with neighboring cells and migrates via the bloodstream or lymph from the primary site of disease to invade neighboring body structures. The terms also refer to the process of metastasis, which includes, but is not limited to detachment of cancer cells from a primary tumor, intravasation of the tumor cells to circulation, their survival and migration to a distant site, attachment and extravasation into a new site from the circulation, and microcolonization at the distant site, and tumor growth and development at the distant site.

The term “therapeutically effective amount” refers to an amount of an antibody, polypeptide, polynucleotide, small organic molecule, or other drug effective to “treat” a disease or disorder in a subject or mammal. In the case of cancer, the therapeutically effective amount of the drug can reduce the number of cancer cells; retard or stop cancer cell division, reduce or retard an increase in tumor size; inhibit, e.g., suppress, retard, prevent, stop, delay, or reverse cancer cell infiltration into peripheral organs including, for example, the spread of cancer into soft tissue and bone; inhibit, e.g., suppress, retard, prevent, shrink, stop, delay, or reverse tumor metastasis; inhibit, e.g., suppress, retard, prevent, stop, delay, or reverse tumor growth; relieve to some extent one or more of the symptoms associated with the cancer, reduce morbidity and mortality; improve quality of life; or a combination of such effects. To the extent the drug prevents growth and/or kills existing cancer cells, it can be referred to as cytostatic and/or cytotoxic.

Terms such as “treating” or “treatment” or “to treat” or “alleviating” or “to alleviate” refer to both 1) therapeutic measures that cure, slow down, lessen symptoms of, reverse, and/or halt progression of a diagnosed pathologic condition or disorder and 2) prophylactic or preventative measures that prevent and/or slow the development of a targeted pathologic condition or disorder. Thus, those in need of treatment include those already with the disorder; those prone to have the disorder; and those in whom the disorder is to be prevented. A subject is successfully “treated” according to the methods of the present disclosure if the patient shows one or more of the following: a reduction in the number of or complete absence of cancer cells; a reduction in tumor size; or retardation or reversal of tumor growth, inhibition, e.g., suppression, prevention, retardation, shrinkage, delay, or reversal of metastases, e.g., of cancer cell infiltration into peripheral organs including, for example, the spread of cancer into soft tissue and bone; inhibition of, e.g., suppression of, retardation of, prevention of, shrinkage of, reversal of, delay of, or an absence of tumor metastases; inhibition of, e.g., suppression of, retardation of, prevention of, shrinkage of, reversal of, delay of, or an absence of tumor growth; relief of one or more symptoms associated with the specific cancer; reduced morbidity and mortality; improvement in quality of life; or some combination of effects. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, cattle, cows, bears, and so on.

As used herein, phrases such as “a subject that would benefit from administration of an anti-SEMA4D antibody in combination with at least one immune checkpoint inhibitor” and “an animal in need of treatment” includes subjects, such as mammalian subjects, that would benefit from administration of an anti-SEMA4D antibody or antigen-binding fragment thereof in combination with at least one immune checkpoint inhibitor.

As used herein, “human” or “fully human” antibodies include antibodies having the amino acid sequence of a human immunoglobulin and include antibodies isolated from human immunoglobulin libraries or from animals transgenic for one or more human immunoglobulins, as described infra and, for example, in U.S. Pat. No. 5,939,598 by Kucherlapati et al. “Human” or “fully human” antibodies also include antibodies comprising at least the variable domain of a heavy chain, or at least the variable domains of a heavy chain and a light chain, where the variable domain(s) have the amino acid sequence of human immunoglobulin variable domain(s).

“Human” or “fully human” antibodies also include “human” or “fully human” antibodies, as described above, that comprise, consist essentially of, or consist of, variants (including derivatives) of antibody molecules (e.g., the VH regions and/or VL regions) described herein, which antibodies or fragments thereof immunospecifically bind to a SEMA4D polypeptide or fragment or variant thereof. Standard techniques known to those of skill in the art can be used to introduce mutations in the nucleotide sequence encoding a human anti-SEMA4D antibody, including, but not limited to, site-directed mutagenesis and PCR-mediated mutagenesis which result in amino acid substitutions. In certain aspects, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference VH region, VHCDR1, VHCDR2, VHCDR3, VL region, VLCDR1, VLCDR2, or VLCDR3.

In certain embodiments, the amino acid substitutions are conservative amino acid substitution, discussed further below. Alternatively, mutations can be introduced randomly along all or part of the coding sequence, such as by saturation mutagenesis, and the resultant mutants can be screened for biological activity to identify mutants that retain activity (e.g., the ability to bind a SEMA4D polypeptide, e.g., human, murine, or both human and murine SEMA4D). Such variants (or derivatives thereof) of “human” or “fully human” antibodies can also be referred to as human or fully human antibodies that are “optimized” or “optimized for antigen binding” and include antibodies that have improved affinity to antigen.

The terms “antibody” and “immunoglobulin” are used interchangeably herein. An antibody or immunoglobulin comprises at least the variable domain of a heavy chain, and normally comprises at least the variable domains of a heavy chain and a light chain. Basic immunoglobulin structures in vertebrate systems are relatively well understood. See, e.g., Harlow et al. (1988) Antibodies: A Laboratory Manual (2nd ed.; Cold Spring Harbor Laboratory Press).

As used herein, the term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA IgD, or IgE, respectively. The immunoglobulin subclasses (isotypes) e.g., IgG1, IgG2, IgG3, IgG4, IgA1, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VL or VK) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminal portion is a variable region and at the C-terminal portion is a constant region; the CH3 and CL domains actually comprise the carboxy-terminus of the heavy and light chain, respectively.

As indicated above, the variable region allows the antibody to selectively recognize and specifically bind epitopes on antigens. That is, the VL domain and VH domain, or subset of the complementarity determining regions (CDRs) within these variable domains, of an antibody combine to form the variable region that defines a three-dimensional antigen binding site. This quaternary antibody structure forms the antigen binding site present at the end of each arm of the Y. More specifically, the antigen binding site is defined by three CDRs on each of the VH and VL chains. In some instances, e.g., certain immunoglobulin molecules derived from camelid species or engineered based on camelid immunoglobulins, a complete immunoglobulin molecule can consist of heavy chains only, with no light chains. See, e.g., Hamers-Casterman et al., Nature 363:446-448 (1993).

In naturally occurring antibodies, the six “complementarity determining regions” or “CDRs” present in each antigen binding domain are short, non-contiguous sequences of amino acids that are specifically positioned to form the antigen binding domain as the antibody assumes its three-dimensional configuration in an aqueous environment. The remainder of the amino acids in the antigen binding domains, referred to as “framework” regions, show less inter-molecular variability. The framework regions largely adopt a j-sheet conformation and the CDRs form loops that connect, and in some cases form part of, the j-sheet structure. Thus, framework regions act to form a scaffold that provides for positioning the CDRs in correct orientation by inter-chain, non-covalent interactions. The antigen binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable domain by one of ordinary skill in the art, since they have been precisely defined (see below).

In the case where there are two or more definitions of a term that is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al. (1983) U.S. Dept. of Health and Human Services, “Sequences of Proteins of Immunological Interest” and by Chothia and Lesk, J. Mol. Biol. 196:901-917 (1987), which are incorporated herein by reference, where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues that encompass the CDRs as defined by each of the above cited references are set forth below in Table 1 as a comparison. The exact residue numbers that encompass a particular CDR will vary depending on the sequence and size of the CDR. Those skilled in the art can routinely determine which residues comprise a particular CDR given the variable region amino acid sequence of the antibody.

TABLE 1
CDR Definitions¹

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

¹Numbering of all CDR definitions in Table 1 is according to the numbering conventions set forth by Kabat et al. (see below).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al. (1983) U.S. Dept. of Health and Human Services, “Sequence of Proteins of Immunological Interest.” Unless otherwise specified, references to the numbering of specific amino acid residue positions in an anti-SEMA4D antibody or antigen-binding fragment, variant, or derivative thereof of the present disclosure are according to the Kabat numbering system.

Antibodies or antigen-binding fragments, variants, or derivatives thereof of the disclosure include, but are not limited to, polyclonal, monoclonal, multispecific, bispecific, human, humanized, primatized, or chimeric antibodies, single-chain antibodies, epitope-binding fragments, e.g., Fab, Fab′ and F(ab′)₂, Fd, Fvs, single-chain Fvs (scFv), disulfide-linked Fvs (sdFv), fragments comprising either a VL or VH domain, fragments produced by a Fab expression library, and anti-idiotypic (anti-Id) antibodies (including, e.g., anti-Id antibodies to anti-SEMA4D antibodies disclosed herein). ScFv molecules are known in the art and are described, e.g., in U.S. Pat. No. 5,892,019. Immunoglobulin or antibody molecules of the disclosure can be of any type (e.g., IgG, IgE, IgM, IgD, IgA, and IgY), class (e.g., IgG1, IgG2, IgG3, IgG4, IgA1, and IgA2, etc.), or subclass of immunoglobulin molecule.

As used herein, the term “heavy chain portion” includes amino acid sequences derived from an immunoglobulin heavy chain. In certain embodiments, a polypeptide comprising a heavy chain portion comprises at least one of: a VH domain, a CH1 domain, a hinge (e.g., upper, middle, and/or lower hinge region) domain, a CH2 domain, a CH3 domain, or a variant or fragment thereof. For example, a binding polypeptide for use in the disclosure can comprise a polypeptide chain comprising a CH1 domain; a polypeptide chain comprising a CH1 domain, at least a portion of a hinge domain, and a CH2 domain; a polypeptide chain comprising a CH1 domain and a CH3 domain; a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In another embodiment, a polypeptide of the disclosure comprises a polypeptide chain comprising a CH3 domain. Further, a binding polypeptide for use in the disclosure can lack at least a portion of a CH2 domain (e.g., all or part of a CH2 domain). As set forth above, it will be understood by one of ordinary skill in the art that these domains (e.g., the heavy chain portions) can be modified such that they vary in amino acid sequence from the naturally occurring immunoglobulin molecule.

In certain anti-SEMA4D antibodies, or antigen-binding fragments, variants, or derivatives thereof disclosed herein, the heavy chain portions of one polypeptide chain of a multimer are identical to those on a second polypeptide chain of the multimer. Alternatively, heavy chain portion-containing monomers of the disclosure are not identical. For example, each monomer can comprise a different target binding site, forming, for example, a bispecific antibody. A bispecific antibody is an artificial protein that is composed of fragments of two different monoclonal antibodies and consequently binds to two different types of antigen. Variations on the bispecific antibody format are contemplated within the scope of the present disclosure. Bispecific antibodies can be generated using techniques that are well known in the art for example, see, for example, Ghayur et al., Expert Review of Clinical Pharmacology 3.4 (July 2010): p. 491; Lu et al., J. Biological Chemistry Vol. 280, No. 20, p. 19665-19672 (2005); Marvin et al., Acta Pharmacologic Sinica 26(6):649-658 (2005); and Milstein C et al., Nature 1983; 305: 537-40; 30 Brennan M et al., Science 1985; 229: 81-3; Thakur et al., Curr Opin Mol Ther. 2010 June; 12(3):340-9; and U.S. Patent Publication No. 2007/0004909.

The heavy chain portions of a binding molecule for use in the methods disclosed herein can be derived from different immunoglobulin molecules. For example, a heavy chain portion of a polypeptide can comprise a CH1 domain derived from an IgG1 molecule and a hinge region derived from an IgG3 molecule. In another example, a heavy chain portion can comprise a hinge region derived, in part, from an IgG1 molecule and, in part, from an IgG3 molecule. In another example, a heavy chain portion can comprise a chimeric hinge derived, in part, from an IgG1 molecule and, in part, from an IgG4 molecule.

As used herein, the term “light chain portion” includes amino acid sequences derived from an immunoglobulin light chain, e.g., a kappa or lambda light chain. In certain aspects, the light chain portion comprises at least one of a VL or CL domain.

Anti-SEMA4D antibodies, or antigen-binding fragments, variants, or derivatives thereof disclosed herein can be described or specified in terms of the epitope(s) or portion(s) of an antigen, e.g., a target polypeptide disclosed herein (e.g., SEMA4D) that they recognize or specifically bind. The portion of a target polypeptide that specifically interacts with the antigen binding domain of an antibody is an “epitope,” or an “antigenic determinant.” A target polypeptide can comprise a single epitope, but typically comprises at least two epitopes, and can include any number of epitopes, depending on the size, conformation, and type of antigen. Furthermore, it should be noted that an “epitope” on a target polypeptide can be or can include non-polypeptide elements, e.g., an epitope can include a carbohydrate side chain.

The minimum size of a peptide or polypeptide epitope for an antibody is thought to be about four to five amino acids. Peptide or polypeptide epitopes can contain at least seven, at least nine and, in some cases, between at least about 15 to about 30 amino acids. Since a CDR can recognize an antigenic peptide or polypeptide in its tertiary form, the amino acids comprising an epitope need not be contiguous, and in some cases, may not even be on the same peptide chain. A peptide or polypeptide epitope recognized by anti-SEMA4D antibodies of the present disclosure can contain a sequence of at least 4, at least 5, at least 6, at least 7, at least 8, at least 9, at least 10, at least 15, at least 20, at least 25, or between about 15 to about 30 contiguous or non-contiguous amino acids of SEMA4D.

By “specifically binds,” it is generally meant that an antibody binds to an epitope via its antigen binding domain, and that the binding entails some complementarity between the antigen binding domain and the epitope. According to this definition, an antibody is said to “specifically bind” to an epitope when it binds to that epitope, via its antigen binding domain more readily than it would bind to a random, unrelated epitope. The term “specificity” is used herein to qualify the relative affinity by which a certain antibody binds to a certain epitope. For example, antibody “A” can be deemed to have a higher specificity or affinity for a given epitope than antibody “B,” or antibody “A” can be said to bind to epitope “C” with a higher specificity or affinity than it has for related epitope “D.”

By “preferentially binds,” it is meant that the antibody specifically binds to an epitope more readily than it would bind to a related, similar, homologous, or analogous epitope. Thus, an antibody that “preferentially binds” to a given epitope would more likely bind to that epitope than to a related epitope, even though such an antibody can cross-react with the related epitope.

By way of non-limiting example, an antibody can be considered to bind a first epitope preferentially if it binds said first epitope with a dissociation constant (K_{D}) that is less than the antibody's K_{D} for the second epitope. In another non-limiting example, an antibody can be considered to bind a first antigen preferentially if it binds the first epitope with an affinity that is at least one order of magnitude less than the antibody's K_{D} for the second epitope. In another non-limiting example, an antibody can be considered to bind a first epitope preferentially if it binds the first epitope with an affinity that is at least two orders of magnitude less than the antibody's K_{D} for the second epitope.

In another non-limiting example, an antibody can be considered to bind a first epitope preferentially if it binds the first epitope with an off rate (k(off)) that is less than the antibody's k(off) for the second epitope. In another non-limiting example, an antibody can be considered to bind a first epitope preferentially if it binds the first epitope with an affinity that is at least one order of magnitude less than the antibody's k(off) for the second epitope. In another non-limiting example, an antibody can be considered to bind a first epitope preferentially if it binds the first epitope with an affinity that is at least two orders of magnitude less than the antibody's k(off) for the second epitope.

An antibody is said to competitively inhibit binding of a reference antibody to a given epitope if it preferentially binds to that epitope to the extent that it blocks, to some degree, binding of the reference antibody to the epitope. Competitive inhibition can be determined by any method known in the art, for example, competition ELISA assays. An antibody can be said to competitively inhibit binding of the reference antibody to a given epitope by at least 90%, at least 80%, at least 70%, at least 60%, or at least 50%.

As used herein, the term “affinity” refers to a measure of the strength of the binding of an individual epitope with the CDR of an immunoglobulin molecule. See, e.g., Harlow et al. (1988) Antibodies: A Laboratory Manual (Cold Spring Harbor Laboratory Press, 2nd ed.) pages 27-28. As used herein, the term “avidity” refers to the overall stability of the complex between a population of immunoglobulins and an antigen, that is, the functional combining strength of an immunoglobulin mixture with the antigen. See, e.g., Harlow at pages 29-34. Avidity is related to both the affinity of individual immunoglobulin molecules in the population with specific epitopes, and also the valencies of the immunoglobulins and the antigen. For example, the interaction between a bivalent monoclonal antibody and an antigen with a highly repeating epitope structure, such as a polymer, would be one of high avidity.

Anti-SEMA4D antibodies or antigen-binding fragments, variants, or derivatives thereof of the disclosure can also be described or specified in terms of their cross-reactivity. As used herein, the term “cross-reactivity” refers to the ability of an antibody, specific for one antigen, to react with a second antigen; a measure of relatedness between two different antigenic substances. Thus, an antibody is cross reactive if it binds to an epitope other than the one that induced its formation. The cross-reactive epitope generally contains many of the same complementary structural features as the inducing epitope, and in some cases, can actually fit better than the original.

For example, certain antibodies have some degree of cross-reactivity, in that they bind related, but non-identical epitopes, e.g., epitopes with at least 95%, at least 90%, at least 85%, at least 80%, at least 75%, at least 70%, at least 65%, at least 60%, at least 55%, and at least 50% identity (as calculated using methods known in the art and described herein) to a reference epitope. An antibody can be said to have little or no cross-reactivity if it does not bind epitopes with less than 95%, less than 90%, less than 85%, less than 80%, less than 75%, less than 70%, less than 65%, less than 60%, less than 55%, and less than 50% identity (as calculated using methods known in the art and described herein) to a reference epitope. An antibody can be deemed “highly specific” for a certain epitope, if it does not bind any other analog, ortholog, or homolog of that epitope.

Anti-SEMA4D binding molecules, e.g., antibodies or antigen-binding fragments, variants or derivatives thereof of the disclosure can also be described or specified in terms of their binding affinity to a polypeptide of the disclosure, e.g., SEMA4D, e.g., human, murine, or both human and murine SEMA4D. In certain aspects, binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M. In certain embodiments, the anti-SEMA4D binding molecule, e.g., an antibody or antigen binding fragment thereof, of the disclosure binds human SEMA4D with a Kd of about 5×10⁻⁹ to about 6×10⁻⁹. In another embodiment, the anti-SEMA4D binding molecule, e.g., an antibody or antigen binding fragment thereof, of the disclosure binds murine SEMA4D with a Kd of about 1×10⁻⁹ to about 2×10⁻⁹.

As used herein, the term “chimeric antibody” will be held to mean any antibody wherein the immunoreactive region or site is obtained or derived from a first species and the constant region (which can be intact, partial or modified) is obtained from a second species. In some embodiments the target binding region or site will be from a non-human source (e.g., mouse or primate) and the constant region is human.

As used herein, the term “engineered antibody” refers to an antibody in which the variable domain in either the heavy or light chain or both is altered by at least partial replacement of one or more CDRs from an antibody of known specificity and, if necessary, by partial framework region replacement and sequence changing. Although the CDRs can be derived from an antibody of the same class or even subclass as the antibody from which the framework regions are derived, it is envisaged that the CDRs will be derived from an antibody of different class or from an antibody from a different species. An engineered antibody in which one or more “donor” CDRs from a non-human antibody of known specificity is grafted into a human heavy or light chain framework region is referred to herein as a “humanized antibody.” In certain aspects it is not necessary to replace all of the CDRs with the complete CDRs from the donor variable domain to transfer the antigen binding capacity of one variable domain to another. Rather, only those residues that are necessary to maintain the activity of the binding site against the targeted antigen can be transferred.

It is further recognized that the framework regions within the variable domain in a heavy or light chain, or both, of a humanized antibody can comprise solely residues of human origin, in which case these framework regions of the humanized antibody are referred to as “fully human framework regions” (for example, MAb VX15/2503, disclosed in U.S. Patent Appl. Publication No. U.S. 2010/0285036 A1 as MAb 2503, incorporated herein by reference in its entirety). Alternatively, one or more residues of the framework region(s) of the donor variable domain can be engineered within the corresponding position of the human framework region(s) of a variable domain in a heavy or light chain, or both, of a humanized antibody if necessary to maintain proper binding or to enhance binding to the SEMA4D antigen. A human framework region that has been engineered in this manner would thus comprise a mixture of human and donor framework residues and is referred to herein as a “partially human framework region.”

For example, humanization of an anti-SEMA4D antibody can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent or mutant rodent CDRs or CDR sequences for the corresponding sequences of a human anti-SEMA4D antibody. See also U.S. Pat. Nos. 5,225,539; 5,585,089; 5,693,761; 5,693,762; 5,859,205; herein incorporated by reference. The resulting humanized anti-SEMA4D antibody would comprise at least one rodent or mutant rodent CDR within the fully human framework regions of the variable domain of the heavy and/or light chain of the humanized antibody. In some instances, residues within the framework regions of one or more variable domains of the humanized anti-SEMA4D antibody are replaced by corresponding non-human (for example, rodent) residues (see, for example, U.S. Pat. Nos. 5,585,089; 5,693,761; 5,693,762; and 6,180,370), in which case the resulting humanized anti-SEMA4D antibody would comprise partially human framework regions within the variable domain of the heavy and/or light chain. Similar methods can be used for humanization of an anti-VEGF antibody.

Furthermore, humanized antibodies can comprise residues that are not found in the recipient antibody or in the donor antibody. These modifications are made to further refine antibody performance (e.g., to obtain desired affinity). In general, the humanized antibody will comprise substantially all of at least one, and typically two, variable domains, in which all or substantially all of the CDRs correspond to those of a non-human immunoglobulin and all or substantially all of the framework regions are those of a human immunoglobulin sequence. The humanized antibody optionally also will comprise at least a portion of an immunoglobulin constant region (Fc), typically that of a human immunoglobulin. For further details see Jones et al., Nature 331:522-525 (1986); Riechmann et al., Nature 332:323-329 (1988); and Presta, Curr. Op. Struct. Biol. 2:593-596 (1992); herein incorporated by reference. Accordingly, such “humanized” antibodies can include antibodies wherein substantially less than an intact human variable domain has been substituted by the corresponding sequence from a non-human species. In practice, humanized antibodies are typically human antibodies in which some CDR residues and possibly some framework residues are substituted by residues from analogous sites in rodent antibodies. See, for example, U.S. Pat. Nos. 5,225,539; 5,585,089; 5,693,761; 5,693,762; 5,859,205. See also U.S. Pat. No. 6,180,370, and International Publication No. WO 01/27160, where humanized antibodies and techniques for producing humanized antibodies having improved affinity for a predetermined antigen are disclosed.

### II. Determination of PD-L1 Status and Methods of Selecting Subjects for Treatment

The present disclosure relates to the use of PD-L1 status, and in particular, low PD-L1 expression as found on tumor cells, cancer cells or tumor tissue, to select subjects who will benefit from a combination cancer immunotherapy of the disclosure. The combination cancer immunotherapy for the selected subjects is a combination of an anti-SEMA4D antibody (or antigen binding fragments, variants and derivatives thereof) and at least one other immune modulating therapy, where the other immune modulating therapy is preferably an immune checkpoint inhibitor.

Accordingly, one aspect of this disclosure provides a method of selecting subjects having or suspected of having cancer for treatment with an isolated antibody, or antigen-binding fragment thereof, that specifically binds to semaphorin-4D (SEMA4D) and an effective amount of at least one other immune modulating therapy, preferably at least one immune checkpoint inhibitor, which method comprises (a) determining PD-L1 expression levels in tumor tissue of a subject; and (b) selecting the subject for treatment if the tumor tissue is identified as having a combined positive score (CPS) of <25 for PD-L1 expression, wherein the treatment is a combination immunotherapy of the disclosure. In some embodiments, the tumor tissue is identified as having a CPS of <20. In some embodiments, the tumor tissue is identified as having a CPS of <10.

The combination immunotherapy treatments of the disclosure are more fully discussed in Section IV hereof.

“PD-L1” expression means any detectable level of expression of PD-L1 on the cell surface or of PD-L1 mRNA within a cell or tissue, unless otherwise defined. For example, PD-L1 expression may be detected with a diagnostic PD-L1 antibody in an IHC assay of a tumor tissue section or by flow cytometry (especially for bodily fluids obtained from subjects with hematological cancers, for example). Alternatively, PD-L1 expression by tumor cells may be detected by PET imaging, using a binding agent (e.g., antibody fragment, affibody and the like) that specifically binds to PD-L1. Techniques for detecting and measuring PD-L1 mRNA expression include RT-PCR and real-time quantitative RT-PCR.

Methods for determining PD-L1 expression are well known in the art, with immunohistochemical (IHC) staining of paraffin-imbedded tumor tissue using an anti-PD-L1 antibody being widely employed.

Several approaches have been described for quantifying PD-L1 protein expression in IHC assays of tumor tissue sections. See, e.g., Thompson et al., PNAS 101: 17174-17179 (2004); Thompson et al., Cancer Res. 66:3381-3385 (2006); Gadiot et al., Cancer 117:2192-2201 (2011); Taube et al., Sci Transl Med 4, 127ra37 (2012); and Toplian et al., New Eng. J Med. 366: 2443-2454 (2012).

IHC results can be scored by assessing positive expression of PD-L1 in tumor cells (TC) or tumor cells and immune cells (IC) present in a sample. The results are reported as CPS or TPS. When cells present in bodily fluids are analyzed by FACS using anti-PD-L1 antibodies, the results are generally reported as TPS.

Many different IHC assays useful for PD-L1 expression are known and the choices of which to use may vary based on the anti-PD-L1 monoclonal antibody selected for the staining process (e.g., PD-L1 28-8, PD-L1 22C3, PD-L1 SP142, and PD-L1 SP263) and the type of tumor tissue being stained.

Specific examples of diagnostic anti-human PD-L1 mAbs useful for IHC detection of PD-L1 expression in formalin-fixed, paraffin-embedded (FFPE) tumor tissue sections are antibody 20C3 and antibody 22C3, which are described in WO 2014/100079.

Another anti-human PD-L1 mAb that has been reported to be useful for IHC detection of PD-L1 expression in FFPE tissue sections (Chen, B. J. et al., Clin Cancer Res 19:3462-3473 (2013)) is a rabbit anti-human PD-L1 mAb publicly available from Sino Biological, Inc. (Beijing, P.R. China; Catalog number 10084-R015).

In many instances, FDA approved companion and complementary assays have been established for specific cancers and the immunotherapy therapeutic being used (see Table 2). Such FDA approved companion diagnostic tests can be used to identify and select patients in accordance with the disclosure. An example of one such IHC assay is PD-L1 IHC 22C3 pharmDx which uses monoclonal mouse Anti-PD-L1 Clone 22C3 for detection of PD-L1 protein in FFPE non-small cell lung cancer (NSCLC), gastric or gastroesophageal junction (GEJ) adenocarcinoma, cervical cancer and urothelial carcinoma tissues using EnVision FLEX visualization system on Autostainer Link 48.

TABLE 2
Exemplary PD-L1 expression IHC Assays

| PDL1 | | | |
|---|---|---|---|
| mAb | | PD-L1 | |
| clone | Immunotherapy | Scoring | PD-L1 Low Cutoffs |
| 22C3 | Pembrolizumab | TC, IC | TPS <50 (NSCLC: KN-189, KN-042) |
| | (PD-1) | | TPS <50 (HNSCC: KN-040) |
| | | | CPS <20 (HNSCC: KN-048), |
| | | | CPS <10 (Gastric: KN-062) |
| | | | CPS <10 (Bladder: KN-045) |
| | | | CPS <10 (Urothelial: KN-052) |
| 28-8 | Nivolumab (PD-1) | TC | TPS <1% (Urothelial: CHECKMATE-275) |
| SP142 | Atezolizumab | TC, IC | TC <3, IC <3 (NSCLC: Impower-130, Impower-131) |
| | (PDL1) | | IC <2, PDL1 <5% (Urothelial: Imvigor-210) |
| SP263 | Durvalumab (PDL1) | TC | Urothelial <25% |
| 73-10 | Avelumab (PDL1) | TC | TPS <80% (NSCLC) |

Abbreviations: mAb, monoclonal antibody; TC: tumor cell; IC: immune cells; KN: KEYNOTE study; NSCLC: non small cell lung carcinoma; HNSCC: head and neck squamous cell carcinoma.

For the methods hereof, PD-L1 expression is scored by CPS or TPS. As used herein, “combined positive score” “Combined Positive Score” or “CPS” refers to a scoring method that counts the number of PD-L1 positive cells in a sample for the tumor cells, lymphocytes and macrophages in the sample relative to the total number of viable tumor cells in that sample multiplied by 100. Although the result of a CPS calculation can exceed 100, the maximum score is defined as CPS 100. A minimum of 100 viable tumor cells in the sample is usually required for a specimen to be considered adequate for PD-L1 evaluation.

As used herein, “tumor proportion score,” “Tumor Proportion Score,” and “TPS” refers to a scoring method that is based on the proportion of tumor cells that express PD-L1 relative to the total number of tumor cells (staining and non-staining). TPS is determined as the percentage of tumor cells showing partial or complete membrane staining at any intensity.

The cutoff point between low PD-L1 and high PD-L1 expression is known for many PD-L1 expression assays as shown, for example, in Table 1, last column. In general, a specimen is considered to have low PD-L1 expression if TPS <50% and high PD-L1 expression if TPS ≥50%.

Hence, as used herein, “PDL1 Low,” “PD-L1 Low,” “PD-L1 Low status,” “low PD-L1 expression,” and grammatical variations on these terms, means the tumor tissue has a CPS of <25 for PD-L1 expression. In some embodiments, the tumor tissue is identified as having a lower CPS, e.g., <20 or even <10. In terms of TPS, PD-L1 Low ranges from <50% to <80%. In some embodiments, the tumor tissue or cancerous cells are identified as having a TPS <50%.

For example, with the 22C3 IHC assay, PD-L1 Low is a CPS <20 or TPS <50% in NSCLC or HSNCC. With the 73-10 IHC assay, PD-L1 Low is TPS <80% in NSCLC.

In select embodiments of any of the methods and uses described herein, the cancer for treatment is esophageal cancer and the CPS for PD-L1 expression is <25. In some of these embodiments the cancer is locally advanced, recurrent or metastatic esophageal cancer.

In select embodiments of any of the methods and uses described herein, the cancer for treatment is an HNSCC, or other head and neck cancer, and the CPS for PD-L1 expression is <20. In some of these embodiments the cancer is locally advanced, recurrent or metastatic HNSCC or other head and neck cancer.

In select embodiments of any of the methods and uses described herein, the cancer for treatment is a lung cancer, and the CPS for PD-L1 expression is <10. In some of these embodiments the cancer is locally advanced, recurrent or metastatic lung cancer. In some of these embodiments, lung cancer is non-small cell lung cancer.

In select embodiments of any of the methods and uses described herein, the cancer for treatment is a lung cancer, and the TPS for PD-L1 expression is <80%. In some of these embodiments the cancer is locally advanced, recurrent or metastatic lung cancer. In some of these embodiments, lung cancer is non-small cell lung cancer.

TPS<80 (using 73-10 assay) in NSCLC.

Cancer specimens that can be assessed for PD-L1 expression include, but are not limited to, tumor tissue obtained by biopsy (or other method) or to cancerous cells as found in or obtained from a bodily fluid such as blood, sera, lymphatic fluid and the like.

In some embodiments, the methods for selecting subjects having or suspected of having cancer for treatment, the anti-SEMA4D antibody or antigen-binding fragment thereof are those described in Section III herein.

In some embodiments, the methods for selecting subjects having or suspected of having cancer for treatment, the immune modulating therapy is as described in Section IV herein. In some embodiments, the immune modulating therapy uses at least one immune checkpoint inhibitor. In some embodiments, the immune checkpoint inhibitor is an anti-CTLA4 antibody, an anti-PD-1 antibody, an anti-PD-L1 antibody, an anti-LAG3 antibody, or a combination thereof. In some embodiments, the anti-PD-1 antibody is pembrolizumab, cemiplimab, dostarlimab, or nivolumab. In some embodiments, the anti-PD-L1 antibody is atezolizumab, durvalumab, avelumab. In some embodiments, the anti-LAG3 antibody is relatlimab (Opduolag, combination with nivolumab). In some embodiments, the anti-PD-1 antibody is pembrolizumab.

The provided methods of the disclosure can be used to select and treat subjects with any cancer, e.g., a solid tumor, a hematological malignancy, any metastasis thereof, or any combination thereof. In certain aspect the solid tumor is a sarcoma, a carcinoma, a melanoma, any metastases thereof, or any combination thereof. In certain aspects the solid tumor can be squamous cell carcinoma, adenocarcinoma, basal cell carcinoma, renal cell carcinoma, ductal carcinoma of the breast, soft tissue sarcoma, osteosarcoma, melanoma, small-cell lung cancer, non-small cell lung cancer, adenocarcinoma of the lung, cancer of the peritoneum, hepatocellular carcinoma, gastrointestinal cancer, gastric cancer, pancreatic cancer, neuroendocrine cancer, glioblastoma, cervical cancer, ovarian cancer, bladder cancer, brain cancer, hepatoma, breast cancer, colon cancer, colorectal cancer, endometrial or uterine carcinoma, esophageal cancer, salivary gland carcinoma, kidney cancer, liver cancer, prostate cancer, vulval cancer, thyroid cancer, head and neck cancer, any metastases thereof, or any combination thereof. In certain aspects the cancer is non-small cell lung cancer. In certain aspects the hematologic malignancy is leukemia, lymphoma, myeloma, acute myeloid leukemia, chronic myeloid leukemia, acute lymphocytic leukemia, chronic lymphocytic leukemia, hairy cell leukemia, Hodgkin lymphoma, non-Hodgkin lymphoma, multiple myeloma, any metastases thereof, or any combination thereof.

In some embodiments, the cancer is locally advanced or recurrent or metastatic (in all of the following) head and neck cancer, non-small cell lung cancer, cervical cancer, urothelial carcinoma, esophageal squamous cell carcinoma (ESCC), renal cell carcinoma, ovarian cancer, prostate cancer, bladder cancer, pancreatic cancer, gastrointestinal cancer, hepatocellular carcinoma, sarcoma, melanoma, triple negative breast cancer (TNBC), microsatellite instability/mismatch repair (MSI/MMR) deficient tumors and colorectal cancer.

### III. Anti-SEMA4D Antibodies

Antibodies that bind SEMA4D have been described in the art. See, for example, U.S. Pat. Nos. 8,496,938; 11,427,634, U.S. Publ. Nos. 2008/0219971, U.S. 2010/0285036, and U.S. 2006/0233793, U.S. Publ. No. 2021/0032329, International Patent Applications WO 93/14125, WO 2008/100995, and WO 2010/129917, and Herold et al., Int. Immunol. 7(1): 1-8 (1995), each of which is herein incorporated in its entirety by reference.

In certain embodiments, the antibody blocks the interaction of SEMA4D with one or more of its receptors, e.g., Plexin-B1, Plexin-B2, and CD72. In certain embodiments the cancer cells and tumor infiltrating immune cells express Plexin-B1, Plexin-B2 and/or CD72. Anti-SEMA4D antibodies having these properties can be used in the methods provided herein. Antibodies that can be used include but are not limited to MAbs VX15/2503 (pepinemab), 67, 76, 2282, VX18, D2517, or D2585, and antigen-binding fragments, variants, or derivatives thereof which are fully described in U.S. 2010/0285036, U.S. 2008/0219971 and U.S. Pat. No. 11,427,634. The amino acid sequences of the VH and VL regions, as well as the associated CDRs for pepinemab (with VH/VL SEQ ID NOS:7 and 8) and VX18 are provided in Table 3A and Table 3B, respectively.

TABLE 3A

| SEQ ID | | |
|---|---|---|
| NO: | Region | SEQUENCE (MAbVX15/2503) |
| 1 | VH CDR1 | Gly Tyr Ser Phe Ser Asp Tyr Tyr Met His |
| 2 | VH CDR2 | Gln Ile Asn Pro Thr Thr Gly Gly Ala Ser Tyr Asn Gln Lys Phe Lys |
| 3 | VH CDR3 | Tyr Tyr Tyr Gly Arg His Phe Asp Val |
| 4 | VL CDR1 | Lys Ala Ser Gln Ser Val Asp Tyr Asp Gly Asp Ser Tyr Met Asn |
| 5 | VL CDR2 | Ala Ala Ser Asn Leu Glu Ser |
| 6 | VL CDR3 | Gln Gln Ser Asn Glu Asp Pro Tyr Thr |
| 7 | VH | Gln Val Gln Leu Val Gln Ser Gly Ala Glu Val Lys Lys Pro Gly Ser Ser Val |
| | human | Lys Val Ser Cys Lys Ala Ser Gly Tyr Ser Phe Ser Asp Tyr Tyr Met His Trp |
| | | Val Arg Gln Ala Pro Gly Gln Gly Leu Glu Trp Met Gly Gln Ile Asn Pro Thr |
| | | Thr Gly Gly Ala Ser Tyr Asn Gln Lys Phe Lys Gly Lys Ala Thr Ile Thr Val |
| | | Asp Lys Ser Thr Ser Thr Ala Tyr Met Glu Leu Ser Ser Leu Arg Ser Glu Asp |
| | | Thr Ala Val Tyr Tyr Cys Ala Arg Tyr Tyr Tyr Gly Arg His Phe Asp Val Trp |
| | | Gly Gln Gly Thr Thr Val Thr Val Ser Ser |
| 8 | VL | Asp Ile Val Met Thr Gln Ser Pro Asp Ser Leu Ala Val Ser Leu Gly Glu Arg |
| | human | Ala Thr Ile Asn Cys Lys Ala Ser Gln Ser Val Asp Tyr Asp Gly Asp Ser Tyr |
| | | Met Asn Trp Tyr Gln Gln Lys Pro Gly Gln Pro Pro Lys Leu Leu Ile Tyr Ala |
| | | Ala Ser Asn Leu Glu Ser Gly Val Pro Asp Arg Phe Ser Gly Ser Gly Ser Gly |
| | | Thr Asp Phe Thr Leu Thr Ile Ser Ser Leu Gln Ala Glu Asp Val Ala Val Tyr |
| | | Tyr Cys Gln Gln Ser Asn Glu Asp Pro Tyr Thr Phe Gly Gln Gly Thr Lys Leu |
| | | Glu Ile Lys |
| 9 | VH | Gln Val Gln Leu Gln Gln Ser Gly Pro Glu Leu Val Lys Pro Gly Ala Ser Val |
| | mouse | Lys Ile Ser Cys Lys Ala Ser Gly Tyr Ser Phe Ser Asp Tyr Tyr Met His Trp |
| | | Val Lys Gln Ser Pro Glu Asn Ser Leu Glu Trp Ile Gly Gln Ile Asn Pro Thr |
| | | Thr Gly Gly Ala Ser Tyr Asn Gln Lys Phe Lys Gly Lys Ala Thr Leu Thr Val |
| | | Asp Lys Ser Ser Ser Thr Ala Tyr Met Gln Leu Lys Ser Leu Thr Ser Glu Glu |
| | | Ser Ala Val Tyr Tyr Cys Thr Arg Tyr Tyr Tyr Gly Arg His Phe Asp Val Trp |
| | | Gly Gln Gly Thr Thr Val Thr Val Ser Ser |
| 10 | VL | Asp Ile Val Met Thr Gln Ser Pro Ala Ser Leu Ala Val Ser Leu Gly Gln Arg |
| | mouse | Ala Thr Ile Ser Cys Lys Ala Ser Gln Ser Val Asp Tyr Asp Gly Asp Ser Tyr |
| | | Met Asn Trp Tyr Gln Gln Lys Pro Gly Gln Pro Pro Lys Leu Leu Ile Tyr Ala |
| | | Ala Ser Asn Leu Glu Ser Gly Ile Pro Ala Arg Phe Ser Gly Ser Gly Ser Gly |
| | | Thr Asp Phe Thr Leu Asn Ile His Pro Val Glu Glu Glu Asp Ala Ala Thr Tyr |
| | | Tyr Cys Gln Gln Ser Asn Glu Asp Pro Tyr Thr Phe Gly Gly Gly Thr Lys Leu |
| | | Glu Ile Lys |

TABLE 3B

| SEQ | | |
|---|---|---|
| ID NO: | Region | SEQUENCE VX18 (MAb D2517) |
| 11 | VH CDR1 | Asp Tyr Trp Met Val |
| 12 | VH CDR2 | His Met Asn Gln Asp Gly Gly Ala Arg Tyr Tyr |
| 13 | VH CDR3 | Asp Pro Trp Gly Tyr |
| 14 | VL CDR1 | Ser Gly Asp Lys Leu Gly Asp Lys Tyr Ala Val |
| 15 | VL CDR2 | Gln Asp Ser Lys Arg Pro Ser |
| 16 | VL CDR3 | Gn Ala Trp Glu Gln Glu Ala Ala Trp Val |
| 17 | VH | Glu Val Gln Leu Val Glu Ser Gly Gly Gly Leu Val Gln Pro Gly Gly Ser Leu |
| | | Arg Leu Ser Cys Ala Ala Ser Gly Phe Ile Phe Ser Asp Tyr Trp Met Val Trp |
| | | Val Arg Gln Ala Pro Gly Lys Gly Leu Glu Tyr Val Ala His Met Asn Gln Asp |
| | | Gly Gly Ala Arg Tyr Tyr Ala Glu Ser Val Arg Gly Arg Phe Thr Ile Ser Arg |
| | | Asp Asn Ala Lys Asn Ser Leu Tyr Leu Gln Met Asn Ser Leu Arg Ala Glu |
| | | Asp Thr Ala Val Tyr Tyr Cys Ala Arg Asp Pro Trp Gly Tyr Asp Ser Trp Gly |
| | | Gln Gly Thr Leu Val Thr |
| 18 | VL | Ser Tyr Glu Leu Thr Gln Pro Pro Ser Val Ser Val Ser Pro Gly Gln Thr Ala |
| | | Ser Ile Thr Cys Ser Gly Asp Lys Leu Gly Asp Lys Tyr Ala Val Trp Tyr Gln |
| | | Gln Lys Pro Gly Gln Ser Pro Val Leu Val Ile Tyr Gln Asp Ser Lys Arg Pro |
| | | Ser Gly Ile Pro Glu Arg Phe Ser Gly Ser Asn Ser Gly Asn Thr Ala Thr Leu |
| | | Thr Ile Ser Gly Thr Gln Ala Met Asp Glu Ala Asp Tyr Tyr Cys Gln Ala Trp |
| | | Glu Gln Glu Ala Ala Trp Val Phe Gly Gly Gly Thr Lys Leu |

Additional antibodies which can be used in the methods provided herein include the BD16 antibody described in U.S. 2006/0233793 A1 as well as antigen-binding fragments, variants, or derivatives thereof; or any of MAb 301, MAb 1893, MAb 657, MAb 1807, MAb 1656, MAb 1808, Mab 59, MAb 2191, MAb 2274, MAb 2275, MAb 2276, MAb 2277, MAb 2278, MAb 2279, MAb 2280, MAb 2281, MAb 2282, MAb 2283, MAb 2284, and MAb 2285, as well as any fragments, variants or derivatives thereof as described in U.S. 2008/0219971 A1. In certain embodiments an anti-SEMA4D antibody for use in the methods provided herein binds human, murine, or both human and murine SEMA4D. Also useful are antibodies which bind to the same epitope as any of the aforementioned antibodies and/or antibodies which competitively inhibit binding or activity of any of the aforementioned antibodies.

In certain embodiments, an anti-SEMA4D antibody or antigen-binding fragment, variant, or derivative thereof useful in the methods provided herein has an amino acid sequence that has at least about 80%, about 85%, about 88%, about 89%, about 90%, about 91%, about 92%, about 93%, about 94%, or about 95% sequence identity to the amino acid sequence for a reference anti-SEMA4D antibody molecule, for example, those described above. In a further embodiment, the binding molecule shares at least about 96%, about 97%, about 98%, about 99%, or 100% sequence identity to a reference antibody.

In another embodiment, an anti-SEMA4D antibody or antigen-binding fragment, variant, or derivative thereof useful in the methods provided herein comprises, consists essentially of, or consists of an immunoglobulin heavy chain variable domain (VH domain), where at least one of the CDRs of the VH domain has an amino acid sequence that is at least about 80%, about 85%, about 90%, about 95%, about 96%, about 97%, about 98%, about 99%, or identical to any CDR1, CDR2 or CDR3 set forth in Tables 3A and 3B.

In another embodiment, an anti-SEMA4D antibody or antigen-binding fragment, variant, or derivative thereof useful in the methods provided herein comprises, consists essentially of, or consists of an immunoglobulin heavy chain variable domain (VH domain), where at least one of the CDRs of the VH domain has an amino acid sequence identical, except for 1, 2, 3, 4, or 5 conservative amino acid substitutions, to any CDR1, CDR2 or CDR3 set forth in Tables 3A and 3B.

In another embodiment, an anti-SEMA4D antibody or antigen-binding fragment, variant, or derivative thereof useful in the methods provided herein comprises, consists essentially of, or consists of a VH domain that has an amino acid sequence that is at least about 80%, about 85%, about 90%, about 91%, about 92%, about 93%, about 94%, about 95%, about 96%, about 97%, about 98%, about 99%, or 100% identical to those VH domains set forth in Tables 3A and 3B, wherein an anti-SEMA4D antibody comprising the encoded VH domain specifically or preferentially binds to SEMA4D.

In another embodiment, an anti-SEMA4D antibody or antigen-binding fragment, variant, or derivative thereof useful in the methods provided herein comprises, consists essentially of, or consists of an immunoglobulin light chain variable domain (VL domain), where at least one of the CDRs of the VL domain has an amino acid sequence that is at least about 80%, about 85%, about 90%, about 95%, about 96%, about 97%, about 98%, about 99%, or identical to any CDR1, CDR2 or CDR3 set forth in Tables 3A and 3B.

In another embodiment, an anti-SEMA4D antibody or antigen-binding fragment, variant, or derivative thereof useful in the methods provided herein comprises, consists essentially of, or consists of an immunoglobulin light chain variable domain (VL domain), where at least one of the CDRs of the VL domain has an amino acid sequence identical, except for 1, 2, 3, 4, or 5 conservative amino acid substitutions, to any CDR1, CDR2 or CDR3 set forth in Tables 3A and 3B.

In a further embodiment, an anti-SEMA4D antibody or antigen-binding fragment, variant, or derivative thereof useful in the methods provided herein comprises, consists essentially of, or consists of a VL domain that has an amino acid sequence that is at least about 80%, about 85%, about 90%, about 91%, about 92%, about 93%, about 94%, about 95%, about 96%, about 97%, about 98%, about 99%, or 100% identical to those VL domains set forth in Tables 3A and 3B, wherein an anti-SEMA4D antibody comprising the encoded VL domain specifically or preferentially binds to SEMA4D.

Suitable biologically active variants of the anti-SEMA4D antibodies of the disclosure can be used in the methods of the present disclosure. Such variants will retain the desired binding properties of the parent anti-SEMA4D antibody. Methods for making antibody variants are generally available in the art.

Methods for mutagenesis and nucleotide sequence alterations are well known in the art. See, for example, Walker and Gaastra, eds. (1983) Techniques in Molecular Biology (MacMillan Publishing Company, New York); Kunkel, Proc. Natl. Acad. Sci. USA 82:488-492 (1985); Kunkel et al., Methods Enzymol. 154:367-382 (1987); Sambrook et al. (1989) Molecular Cloning: A Laboratory Manual (Cold Spring Harbor, N.Y.); U.S. Pat. No. 4,873,192; and the references cited therein; herein incorporated by reference. Guidance as to appropriate amino acid substitutions that do not affect biological activity of the polypeptide of interest can be found in the model of Dayhoff et al. (1978) in Atlas of Protein Sequence and Structure (Natl. Biomed. Res. Found., Washington, D.C.), pp. 345-352, herein incorporated by reference in its entirety. The model of Dayhoff et al. uses the Point Accepted Mutation (PAM) amino acid similarity matrix (PAM 250 matrix) to determine suitable conservative amino acid substitutions. In certain aspects, conservative substitutions, such as exchanging one amino acid with another having similar properties are used. Examples of conservative amino acid substitutions as taught by the PAM 250 matrix of the Dayhoff et al. model include, but are not limited to, Gly↔Ala, Val↔Ile↔Leu, Asp↔Glu, Lys↔Arg, Asn↔Gln, and Phe↔Trp↔Tyr.

In constructing variants of the anti-SEMA4D binding molecule, e.g., an antibody or antigen-binding fragment thereof, polypeptides of interest, modifications are made such that variants continue to possess the desired properties, e.g., being capable of specifically binding to a SEMA4D, e.g., human, murine, or both human and murine SEMA4D, e.g., expressed on the surface of or secreted by a cell and having SEMA4D blocking activity, as described herein. In certain aspects, mutations made in the DNA encoding the variant polypeptide maintain the reading frame and do not create complementary regions that could produce secondary mRNA structure. See EP Patent Application Publication No. 75,444.

Methods for measuring anti-SEMA4D binding molecule, e.g., an antibody or antigen-binding fragment, variant, or derivative thereof, binding specificity include, but are not limited to, standard competitive binding assays, assays for monitoring immunoglobulin secretion by T cells or B cells, T cell proliferation assays, apoptosis assays, ELISA assays, and the like. See, for example, such assays disclosed in WO 93/14125; Shi et al., Immunity 13:633-642 (2000); Kumanogoh et al., J Immunol 169:1175-1181 (2002); Watanabe et al., J Immunol 167:4321-4328 (2001); Wang et al., Blood 97:3498-3504 (2001); and Giraudon et al., J Immunol 172(2):1246-1255 (2004), all of which are herein incorporated by reference.

When discussed herein whether any particular polypeptide, including the constant regions, CDRs, VH domains, or VL domains disclosed herein, is at least about 65%, about 70%, about 75%, about 80%, about 85%, about 90%, about 91%, about 92%, about 93%, about 94%, about 95%, about 96%, about 97%, about 98%, about 99%, or even about 100% identical to another polypeptide, the % identity can be determined using methods and computer programs/software known in the art such as, but not limited to, the BESTFIT program (Wisconsin Sequence Analysis Package, Version 8 for Unix, Genetics Computer Group, University Research Park, 575 Science Drive, Madison, Wis. 53711). BESTFIT uses the local homology algorithm of Smith and Waterman (1981) Adv. Appl. Math. 2:482-489, to find the best segment of homology between two sequences. When using BESTFIT or any other sequence alignment program to determine whether a particular sequence is, for example, 95% identical to a reference sequence according to the present disclosure, the parameters are set, of course, such that the percentage of identity is calculated over the full length of the reference polypeptide sequence and that gaps in homology of up to 5% of the total number of amino acids in the reference sequence are allowed.

For purposes of the present disclosure, percent sequence identity can be determined using the Smith-Waterman homology search algorithm using an affine gap search with a gap open penalty of 12 and a gap extension penalty of 2, BLOSUM matrix of 62. The Smith-Waterman homology search algorithm is taught in Smith and Waterman (1981) Adv. Appl. Math. 2:482-489. A variant can, for example, differ from a reference anti-SEMA4D antibody (e.g., MAb VX15/2503, 67, 76, or 2282) by as few as 1 to 15 amino acid residues, as few as 1 to 10 amino acid residues, such as 6-10, as few as 5, as few as 4, 3, 2, or even 1 amino acid residue.

The constant region of an anti-SEMA4D antibody can be mutated to alter effector function in a number of ways. For example, see U.S. Pat. No. 6,737,056B1 and U.S. Patent Application Publication No. 2004/0132101A1, which disclose Fc mutations that optimize antibody binding to Fc receptors.

In certain anti-SEMA4D antibodies or fragments, variants or derivatives thereof useful in the methods provided herein, the Fc portion can be mutated to decrease effector function using techniques known in the art. For example, the deletion or inactivation (through point mutations or other means) of a constant region domain can reduce Fc receptor binding of the circulating modified antibody thereby increasing tumor localization. In other cases, constant region modifications consistent with the instant disclosure moderate complement binding and thus reduce the serum half-life. Yet other modifications of the constant region can be used to modify disulfide linkages or oligosaccharide moieties that allow for enhanced localization due to increased antigen specificity or antibody flexibility. The resulting physiological profile, bioavailability and other biochemical effects of the modifications, such as tumor localization, biodistribution and serum half-life, can easily be measured and quantified using well known immunological techniques without undue experimentation.

Anti-SEMA4D antibodies for use in the methods provided herein include derivatives that are modified, e.g., by the covalent attachment of any type of molecule to the antibody such that covalent attachment does not prevent the antibody from specifically binding to its cognate epitope. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, etc. Additionally, the derivative can contain one or more non-classical amino acids.

A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity (e.g., the ability to bind an anti-SEMA4D polypeptide, to block SEMA4D interaction with its receptor, or to inhibit, delay, or reduce metastases in a subject, e.g., a cancer patient).

For example, it is possible to introduce mutations only in framework regions or only in CDR regions of an antibody molecule. Introduced mutations can be silent or neutral missense mutations, i.e., have no, or little, effect on an antibody's ability to bind antigen. These types of mutations can be useful to optimize codon usage or improve a hybridoma's antibody production. Alternatively, non-neutral missense mutations can alter an antibody's ability to bind antigen. One of skill in the art would be able to design and test mutant molecules with desired properties such as no alteration in antigen binding activity or alteration in binding activity (e.g., improvements in antigen binding activity or change in antibody specificity). Following mutagenesis, the encoded protein can routinely be expressed and the functional and/or biological activity of the encoded protein, (e.g., ability to immunospecifically bind at least one epitope of a SEMA4D polypeptide) can be determined using techniques described herein or by routinely modifying techniques known in the art.

In certain embodiments, the anti-SEMA4D antibodies for use in the methods provided herein comprise at least one optimized complementarity-determining region (CDR). By “optimized CDR” is intended that the CDR has been modified and optimized to improve binding affinity and/or anti-SEMA4D activity that is imparted to an anti-SEMA4D antibody comprising the optimized CDR. “Anti-SEMA4D activity” or “SEMA4D blocking activity” can include activity which modulates one or more of the following activities associated with SEMA4D: B cell activation, aggregation and survival; CD40-induced proliferation and antibody production; antibody response to T cell dependent antigens; T cell or other immune cell proliferation; dendritic cell maturation; demyelination and axonal degeneration; apoptosis of pluripotent neural precursors and/or oligodendrocytes; induction of endothelial cell migration; inhibition of spontaneous monocyte migration; inhibition, delay, or reduction of tumor cell growth or metastasis, binding to cell surface plexin B1 or other receptor, or any other activity association with soluble SEMA4D or SEMA4D that is expressed on the surface of SEMA4D+ cells. In a particular embodiment, anti-SEMA4D activity includes the ability to inhibit, delay, or reduce tumor metastases, either in combination with inhibition, delay, or reduction of primary tumor cell growth and tumor metastases, or independently of primary tumor cell growth and tumor metastases. Anti-SEMA4D activity can also be attributed to a decrease in incidence or severity of diseases associated with SEMA4D expression, including, but not limited to, certain types of cancers including lymphomas, autoimmune diseases, inflammatory diseases including central nervous system (CNS) and peripheral nervous system (PNS) inflammatory diseases, transplant rejections, and invasive angiogenesis. Examples of optimized antibodies based on murine anti-SEMA4D MAb BD16 were described in U.S. Publ. No. 2008/0219971 A1, International Patent Application WO 93/14125 and Herold et al., Int. Immunol. 7(1): 1-8 (1995), each of which are herein incorporated by reference in their entirety. The modifications can involve replacement of amino acid residues within the CDR such that an anti-SEMA4D antibody retains specificity for the SEMA4D antigen and has improved binding affinity and/or improved anti-SEMA4D activity.

### IV. Treatments Using Therapeutic Anti-SEMA4D Antibodies in Combination With at Least One Immune Checkpoint Inhibitor and, Optionally, at Least One Immune Modulating Therapy

Certain methods of the disclosure are directed to the use of anti-SEMA4D antibodies, including antigen-binding fragments, variants, and derivatives thereof, in combination with at least one other immune modulating therapy, to inhibit, delay, or reduce tumor growth or metastases in a subject in need of such inhibition, delay, or reduction, e.g., a cancer patient. More particularly, such methods of the disclosure for treating, inhibiting, delaying, or reducing malignant cell growth in a subject with cancer comprise (a) determining PD-L1 expression levels in tumor tissue from said cancer of the subject or from a bodily fluid of said subject, e.g., blood for a hematological cancer); and (b) if the CPS is <25 administering to the subject an effective amount of an isolated antibody or antigen-binding fragment thereof that specifically binds to semaphorin-4D (SEMA4D) and an effective amount of at least one immune checkpoint inhibitor or at least one other immune modulating therapy, thereby treating the subject. In any of the embodiments herein, the CPS can be <20 or <10. As an alternative to CPS, the method can use TPS as the determinant for low PD-L1 expression. In those embodiments, TPS ranges from <50% to <80% and is preferably <50%. In any of the embodiments herein, PD-L1 expression levels can be determined by immunohistochemical analysis or by any other method known to those of skill in the art (as more fully discussed in Section II). In any of the embodiments herein, the isolated antibody or antigen-binding fragment thereof that specifically binds to semaphorin-4D (SEMA4D) variants, and derivatives of these antibodies are as described in Section III. In some embodiments herein, at least one immune checkpoint inhibitor is an anti-PD1 antibody, an anti-PD-L1 antibody, an anti-LAG3 antibody, or an anti CTLA4 antibody. In any of the embodiments herein, the anti-PD1 antibody is pembrolizumab, cemiplimab or nivolumab, and preferably is pembrolizumab.

Select methods of the disclosure are directed to treating cancer in a human patient identified as having a tumor with low PD-L1 expression which comprises administering to the patient, as combination immunotherapy, (a) an anti-semaphorin-4D (SEMA4D) antibody, or an antigen binding fragment thereof, and (b) an immune checkpoint inhibitor, wherein the tumor has a combined positive score (CPS) of <25 before the immune checkpoint inhibitor is administered. In some of these embodiments, the CPS is <20. In some embodiments, the CPS is <10.

Select methods of the disclosure are directed to treating cancer in a human patient identified as having a tumor with low PD-L1 expression which comprises administering to the patient, as combination immunotherapy, (a) an anti-semaphorin-4D (SEMA4D) antibody, or an antigen binding fragment thereof, and (b) an immune checkpoint inhibitor, wherein the tumor has a total proportion score (TPS) ranging from <50% to <80% before the immune checkpoint inhibitor is administered. In some of these embodiments, the TPS is <50%.

In some embodiments of any of the methods and uses described herein, the at least other immune modulating therapy is at least one immune checkpoint inhibitor. In any of the embodiments herein, at least one immune checkpoint inhibitor is an anti-PD1 antibody, an anti-PD-L1 antibody, an anti CTLA4 antibody or an anti-LAG3 antibody, or an antigen-binding fragment of any of these antibodies. In any of the embodiments herein, the anti-PD1 antibody is pembrolizumab, cemiplimab or nivolumab, and preferably is pembrolizumab.

In any of the embodiments of any of the methods and uses described herein, the anti-SEMA4D antibody is pepinemab and the anti-PD1 antibody is pembrolizumab.

All of the treatment modalities described herein are based on determining that the tumor tissue or cancer cells from the subject exhibits low PD-L1 expression, i.e., has a CPS at least <25, or a TPS ranging from <50% to <80%. In some embodiments, the CPS may be <20 or even <10. In some embodiments, the TPS may be <50%.

Though the following discussion refers to administration of an anti-SEMA4D antibody, the methods described herein are equally applicable to the antigen-binding fragments, variants, and derivatives of these antibodies that retain the desired properties of the antibodies of the disclosure, e.g., capable of specifically binding SEMA4D, e.g., human, mouse, or human and mouse SEMA4D, having SEMA4D neutralizing activity, and/or blocking the interaction of SEMA4D with its receptors. In embodiments of any of the methods and uses described herein, the anti-SEMA4D, its fragments, variants and derivatives are described in Section III.

In one embodiment, the immune modulating therapy can include cancer vaccines, immunostimulatory agents, adoptive T cell or antibody therapy, and immune checkpoint inhibitors (Lizée et al. 2013. Harnessing the Power of the Immune System to Target Cancer. Annu. Rev. Med. Vol. 64 No. 71-90).

Cancer Vaccines. Cancer vaccines activate the body's immune system and natural resistance to an abnormal cell, such as cancer, resulting in eradication or control of the disease. Cancer vaccines generally consist of a tumor antigen in an immunogenic formulation that activates tumor antigen-specific helper cells and/or CTLs and B cells. Vaccines can be in a variety of formulations, including, but not limited to, dendritic cells, especially autologous dendritic cells pulsed with tumor cells or tumor antigens, heterologous tumor cells transfected with an immune stimulating agent such as GM-CSF, recombinant virus, or proteins or peptides that are usually administered together with a potent immune adjuvant such as CpG.

Immunostimulatory Agents. Immunostimulatory agents act to enhance or increase the immune response to tumors, which is suppressed in many cancer patients through various mechanisms. Immune modulating therapies can target lymphocytes, macrophages, dendritic cells, natural killer cells (NK Cell), or subsets of these cells such as cytotoxic T lymphocytes (CTL) or Natural Killer T (NKT) cells. Because of interacting immune cascades, an effect on one set of immune cells will often be amplified by spreading to other cells, e.g. enhanced antigen presenting cell activity promotes response of T and B lymphocytes. Examples of immunostimulatory agents include, but are not limited to, HER2, cytokines such as G-CSF, GM-CSF and IL-2, cell membrane fractions from bacteria, glycolipids that associate with CD1d to activate Natural Killer T (NKT) cells, CpG oligonucleotides.

Macrophages, myelophagocytic cells of the immune system, are a fundamental part of the innate defense mechanisms, which can promote specific immunity by inducing T cell recruitment and activation. Despite this, their presence within the tumor microenvironment has been associated with enhanced tumor progression and shown to promote cancer cell growth and spread, angiogenesis and immunosuppression. Key players in the setting of their phenotype are the microenvironmental signals to which macrophages are exposed, which selectively tune their functions within a functional spectrum encompassing the M1 (tumor inhibiting macrophage) and M2 (tumor promoting macrophage) extremes. Sica et al., Seminars in Cancer Biol. 18:349-355 (2008). Increased macrophage numbers during cancer generally correlates with poor prognosis (Qualls and Murray, Curr. Topics in Develop. Biol. 94:309-328 (2011)). Of the multiple unique stromal cell types common to solid tumors, tumor-associated macrophages (TAMs) are significant for fostering tumor progression. Targeting molecular pathways regulating TAM polarization holds great promise for anticancer therapy. Ruffell et al., Trends in Immunol. 33:119-126 (2012).

Adoptive Cell Transfer. Adoptive cell transfer can employ T cell-based cytotoxic responses to attack cancer cells. Autologous T cells that have a natural or genetically engineered reactivity to a patient's cancer are generated and expanded in vitro and then transferred back into the cancer patient. One study demonstrated that adoptive transfer of in vitro expanded autologous tumor-infiltrating lymphocytes was an effective treatment for patients with metastatic melanoma. (Rosenberg S A, Restifo N P, Yang J C, Morgan R A, Dudley M E (April 2008). “Adoptive cell transfer: a clinical path to effective cancer immunotherapy”. Nat. Rev. Cancer 8 (4): 299-308). This can be achieved by taking T cells that are found within resected patient tumor. These T cells are referred to as tumor-infiltrating lymphocytes (TIL) and are presumed to have trafficked to the tumor because of their specificity for tumor antigens. Such T cells can be induced to multiply in vitro using high concentrations of IL-2, anti-CD3 and allo-reactive feeder cells. These T cells are then transferred back into the patient along with exogenous administration of IL-2 to further boost their anti-cancer activity. In other studies, autologous T cells have been transduced with a chimeric antigen receptor that renders them reactive to a targeted tumor antigen (Liddy et al., Nature Med. 18:980-7, (2012); Grupp et al., New England J. Med. 368:1509-18, (2013)).

Other adoptive cell transfer therapies employ autologous dendritic cells exposed to natural or modified tumor antigens ex vivo that are re-infused into the patient. Provenge is such an FDA approved therapy in which autologous cells are incubated with a fusion protein of prostatic acid phosphatase and GM-CSF to treat patients with prostate tumors. GM-CSF is thought to promote the differentiation and activity of antigen presenting dendritic cells (Small et al., J. Clin. Oncol. 18: 3894-903(2000); U.S. Pat. No. 7,414,108)).

Immune Checkpoint Inhibitors. Immune checkpoint inhibitors enhance T-cell immunity by removing a negative feedback control that limits ongoing immune responses. These types of therapies target inhibitory pathways in the immune system that are crucial for modulating the duration and amplitude of physiological immune responses in peripheral tissues (anti-CTLA4) or in tumor tissue expressing PD-L1 (anti-PD-1 or anti-PD-L1) to minimize collateral tissue damage. Tumors can evolve to exploit certain immune-checkpoint pathways as a major mechanism of immune resistance against T cells that are specific for tumor antigens. Since many immune checkpoints are initiated by ligand-receptor interactions, these checkpoints can be blocked by antibodies to either receptor or ligand or can be modulated by soluble recombinant forms of the ligands or receptors. Neutralization of immune checkpoints allows tumor-specific T cells to continue to function in the otherwise immunosuppressive tumor microenvironment. Examples of immune checkpoint blockade therapies are those which target cytotoxic T-lymphocyte-associated antigen 4 (CTLA-4), PD-1, its ligand PD-L1, LAG3, TIGIT, and B7-H3.

Cyclophosphamide. Cyclophosphamide, a commonly used chemotherapeutic agent, can enhance immune responses. Cyclophosphamide differentially suppresses the function of regulatory T cells (Tregs) relative to effector T cells. Tregs are important in regulating anticancer immune responses. Tumor-infiltrating Tregs have previously been associated with poor prognosis. While agents that target Tregs specifically are currently unavailable, cyclophosphamide has emerged as a clinically feasible agent that can preferentially suppress Tregs relative to other T cells and, therefore, allows more effective induction of antitumor immune responses.

Other Immune-Modulating Therapies: In another embodiment, the combination therapy herein (with a SEMA4D antibody or antigen binding fragment, variant, or derivative thereof and at least one other immune-modulating therapy), can be further combined with either standard or low dose chemotherapy, radiation therapy or a second immune-modulating therapy. Although standard chemotherapy is often immunosuppressive, low doses of chemotherapeutic agents such as cyclophosphamide, doxorubicin, and paclitaxel have been shown to enhance responses to vaccine therapy for cancer (Machiels et al., Cancer Res. 61:3689-3697 (2001)). In some cases, chemotherapy can differentially inactivate T regulatory cells (Treg) and myeloid derived suppressor cells (MDSC) that negatively regulate immune responses in the tumor environment. Radiation therapy has been generally employed to exploit the direct tumorcidal effect of ionizing radiation. Indeed, high dose radiation can, like chemotherapy, be immunosuppressive. Numerous observations, however, suggest that under appropriate conditions of dose fractionation and sequencing, radiation therapy can enhance tumor-specific immune responses and the effects of immune modulating agents. One of several mechanisms that contribute to this effect is cross-presentation by dendritic cells and other antigen presenting cells of tumor antigens released by radiation-induced tumor-cell death (Higgins et al., Cancer Biol. Ther. 8:1440-1449 (2009)). In effect, radiation therapy can induce in situ vaccination against a tumor (Ma et al., Seminar Immunol. 22:113-124 (2010)) and this could be amplified by combination with the therapeutic combinations hereof.

In these further embodiments, the second immune modulating therapy can be an immune modulating agent, including, but not limited to, interleukins such as IL-2, IL-7, IL-12; cytokines such as granulocyte-macrophage colony-stimulating factor (GM-CSF), interferons; various chemokines such as CXCL13, CCL26, CXCL7; antagonists of immune checkpoint blockades such as anti-CTLA-4, anti-PD-1, anti-PD-L1, anti-LAG3 and anti-B7-H3; synthetic cytosine phosphate-guanosine (CpG), oligodeoxynucleotides, glucans, modulators of regulatory T cells (Tregs) such as cyclophosphamide, or other immune modulating agents. In one embodiment, the immune modulating agent is an agonist antibody to 4-1BB (CD137). As reported, such agonist antibody to 4-1BB can give rise to a novel class of KLRG1+ T cells that are highly cytotoxic for tumors (Curran et al., J. Exp. Med. 210:743-755 (2013)). In all cases, the additional immune modulating therapy is administered prior to, during, or subsequent to the combination therapy hereof (with anti-SEMA4D antibody or antigen binding fragment, variant, or derivative thereof, in combination with administration of another immune modulating agent). The methods of the disclosure encompass co-administration, using separate formulations or a single pharmaceutical formulation, with simultaneous or consecutive administration in either order.

Where the combined therapies comprise administration of an anti-SEMA4D antibody or antigen binding fragment, variant, or derivative thereof, in combination with administration of another therapeutic agent, the methods of the disclosure encompass co-administration, using separate formulations or a single pharmaceutical formulation, with simultaneous or consecutive administration in either order.

In one embodiment, treatment includes the application or administration of an anti-SEMA4D antibody or antigen binding fragment thereof as described herein in combination with at least one other immune modulating therapy to a patient, or application or administration of the anti-SEMA4D binding molecule in combination with at least one other immune modulating therapy to an isolated tissue or cell line from a patient, where the patient has, or has the risk of developing metastases of cancer cells.

The anti-SEMA4D antibodies or binding fragments thereof as described herein, in combination with at least one other immune modulating therapy are useful for the treatment of various malignant and non-malignant tumors. By “anti-tumor activity” is intended a reduction in the rate of SEMA4D production or accumulation associated directly with the tumor or indirectly with stromal cells of the tumor environment, and hence a decline in growth rate of an existing tumor or of a tumor that arises during therapy, and/or destruction of existing neoplastic (tumor) cells or newly formed neoplastic cells, and hence a decrease in the overall size of a tumor and/or the number of metastatic sites during therapy. For example, therapy with at least one anti-SEMA4D antibody in combination with at least one other immune modulating therapy causes a physiological response, for example, a reduction in metastases, that is beneficial with respect to treatment of disease states associated with SEMA4D-expressing cells in a human.

In one embodiment, the disclosure relates to the use of anti-SEMA4D antibodies or antigen-binding fragments, variants, or derivatives thereof, in combination with at least one other immune modulating therapy as a medicament, in the treatment or prophylaxis of cancer or for use in a precancerous condition or lesion to inhibit, reduce, prevent, delay, or minimalize the growth or metastases of tumor cells.

In accordance with the methods of the present disclosure, at least one anti-SEMA4D antibody or antigen binding fragment, variant, or derivative thereof, in combination with at least one other immune modulating therapy can be used to promote a positive therapeutic response with respect to a malignant human cell. By “positive therapeutic response” with respect to cancer treatment is intended an improvement in the disease in association with the anti-tumor activity of these binding molecules, e.g., antibodies or fragments thereof, and/or an improvement in the symptoms associated with the disease. In particular, the methods provided herein are directed to inhibiting, preventing, reducing, alleviating, delaying, or lessening growth of a tumor and/or the development of metastases of primary tumors in a patient. That is the prevention of distal tumor outgrowths, can be observed. Thus, for example, an improvement in the disease can be characterized as a complete response. By “complete response” is intended an absence of clinically detectable metastases with normalization of any previously abnormal radiographic studies, e.g. at the site of the primary tumor or the presence of tumor metastases in bone marrow. Alternatively, an improvement in the disease can be categorized as being a partial response. By “partial response” is intended at least about a 50% decrease in all measurable metastases (i.e., the number of tumor cells present in the subject at a remote site from the primary tumor). Alternatively, an improvement in the disease can be categorized as being relapse free survival or “progression free survival”. By “relapse free survival” is intended the time to recurrence of a tumor at any site. “Progression free survival” is the time before further growth of tumor at a site being monitored can be detected.

Inhibition, delay, or reduction of metastases can be assessed using screening techniques such as imaging, for example, fluorescent antibody imaging, bone scan imaging, and tumor biopsy sampling including bone marrow aspiration (BMA), or immunohistochemistry. In addition to these positive therapeutic responses, the subject undergoing therapy can experience the beneficial effect of an improvement in the symptoms associated with the disease.

Clinical response can be assessed using screening techniques such as magnetic resonance imaging (MRI) scan, x-radiographic imaging, computed tomographic (CT) scan, flow cytometry or fluorescence-activated cell sorter (FACS) analysis, histology, gross pathology, and blood chemistry, including but not limited to changes detectable by ELISA, RIA, chromatography, and the like.

### V. Pharmaceutical Compositions and Administration Methods

Methods of preparing and administering anti-SEMA4D antibodies, or antigen-binding fragments, variants, or derivatives thereof in combination with at least one other immune modulating therapy to a subject in need thereof are well known to or are readily determined by those skilled in the art. The route of administration of the anti-SEMA4D antibody, or antigen-binding fragment, variant, or derivative thereof in combination with at least one other immune modulating therapy, can be, for example, oral, parenteral, by inhalation or topical at the same or different times for each therapeutic agent. The term parenteral as used herein includes, e.g., intravenous, intraarterial, intraperitoneal, intramuscular, subcutaneous, rectal, or vaginal administration. While all these forms of administration are clearly contemplated as being within the scope of the disclosure, an example of a form for administration would be a solution for injection, in particular for intravenous or intraarterial injection or drip. A suitable pharmaceutical composition for injection can comprise a buffer (e.g. acetate, phosphate or citrate buffer), a surfactant (e.g. polysorbate), optionally a stabilizer agent (e.g. human albumin), etc. However, in other methods compatible with the teachings herein, anti-SEMA4D antibodies, or antigen-binding fragments, variants, or derivatives thereof in combination with at least one other immune modulating therapy can be delivered directly to the site of the adverse cellular population thereby increasing the exposure of the diseased tissue to the therapeutic agent.

As discussed herein, anti-SEMA4D antibodies, or antigen-binding fragments, variants, or derivatives thereof in combination with at least one other immune modulating therapy can be administered in a pharmaceutically effective amount for the in vivo treatment of diseases such as neoplastic disorders, including solid tumors. In this regard, it will be appreciated that the disclosed molecules can be formulated to facilitate administration and promote stability of the active agent. In certain embodiments, pharmaceutical compositions in accordance with the present disclosure comprise a pharmaceutically acceptable, non-toxic, sterile carrier such as physiological saline, non-toxic buffers, preservatives and the like. For the purposes of the instant application, a pharmaceutically effective amount of an anti-SEMA4D antibody, or antigen-binding fragment, variant, or derivative thereof, in combination with at least one other immune modulating therapy shall be held to mean an amount sufficient to achieve effective binding to a target and to achieve a benefit, i.e., to inhibit, delay, or reduce metastases in a cancer patient.

The pharmaceutical compositions used in this disclosure comprise pharmaceutically acceptable carriers, including, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include, e.g., water, alcoholic/aqueous solutions, emulsions or suspensions, including saline and buffered media. Pharmaceutically acceptable carriers can include, but are not limited to, 0.01-0.1 M, or 0.05 M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives can also be present such as, for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition can be sterile and should be fluid to the extent that easy syringability exists. It should be stable under the conditions of manufacture and storage and can be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of a certain particle size in the case of dispersion and by the use of surfactants. Suitable formulations for use in the therapeutic methods disclosed herein are described in Remington's Pharmaceutical Sciences (Mack Publishing Co.) 16th ed. (1980).

Prevention of the action of microorganisms can be achieved by various antibacterial and antifungal agents, for example, parabens, chlorobutanol, phenol, ascorbic acid, thimerosal and the like. In certain embodiments, isotonic agents, for example, sugars, polyalcohols, such as mannitol, sorbitol, or sodium chloride can be included in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., an anti-SEMA4D antibody, or antigen-binding fragment, variant, or derivative thereof, in combination with at least one other immune modulating therapy) in a certain amount in an appropriate solvent with one or a combination of ingredients enumerated herein, followed by filtered sterilization. Generally, dispersions are prepared by incorporating the active compound into a sterile vehicle, which contains a basic dispersion medium and the other ingredients from those enumerated above. In the case of sterile powders for the preparation of sterile injectable solutions, methods of preparation can include vacuum drying or freeze-drying, which can yield a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations can be packaged and sold in the form of a kit. Such articles of manufacture can have labels or package inserts indicating that the associated compositions are useful for treating a subject suffering from, or predisposed to a disease or disorder.

Parenteral formulations can be a single bolus dose, an infusion or a loading bolus dose followed with a maintenance dose. These compositions can be administered at specific fixed or variable intervals, e.g., once a day, or on an “as needed” basis.

Certain pharmaceutical compositions can be orally administered in an acceptable dosage form including, e.g., capsules, tablets, aqueous suspensions or solutions. Certain pharmaceutical compositions also can be administered by nasal aerosol or inhalation. Such compositions can be prepared as solutions in saline, employing benzyl alcohol or other suitable preservatives, absorption promoters to enhance bioavailability, and/or other conventional solubilizing or dispersing agents.

The amount of an anti-SEMA4D antibody, or fragment, variant, or derivative thereof, or combination with at least one other immune modulating therapy to be combined with the carrier materials to produce a single dosage form will vary depending upon the host treated and the particular mode of administration. The composition can be administered as a single dose, multiple doses or over an established period of time in an infusion. Dosage regimens also can be adjusted to provide the optimum desired response (e.g., a therapeutic or prophylactic response).

In keeping with the scope of the present disclosure, anti-SEMA4D antibodies, or antigen-binding fragments, variants, or derivatives thereof in combination with at least one other immune modulating therapy can be administered to a human or other animal in accordance with the aforementioned methods of treatment in an amount sufficient to produce a therapeutic effect. The anti-SEMA4D antibodies, or antigen-binding fragments, variants or derivatives thereof in combination with at least one other immune modulating therapy can be administered to such human or other animal in a conventional dosage form prepared by combining the antibody provided herein with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of anti-SEMA4D antibodies, or antigen-binding fragments, variants, or derivatives thereof as provided herein can be used.

By “therapeutically effective dose or amount” or “effective amount” is intended an amount of anti-SEMA4D antibody or antigen binding fragment, variant, or derivative thereof, in combination with at least one other immune modulating therapy that when administered brings about a positive therapeutic response with respect to treatment of a patient with a disease to be treated, e.g., an inhibition, delay, or reduction of metastases in the patient.

Therapeutically effective doses for the compositions of the present disclosure, for the inhibition, delay, or reduction of metastases, vary depending upon many different factors, including means of administration, target site, physiological state of the patient, whether the patient is human or an animal, other medications administered, and whether treatment is prophylactic or therapeutic. In certain embodiments the patient is a human, but non-human mammals including transgenic mammals can also be treated. Treatment dosages can be titrated using routine methods known to those of skill in the art to optimize safety and efficacy.

The amount of anti-SEMA4D antibody or binding fragment, variant, or derivative thereof, administered in combination with at least one other immune modulating therapy is readily determined by one of ordinary skill in the art without undue experimentation given the disclosure of the present disclosure. Factors influencing the mode of administration and the respective amount of anti-SEMA4D antibody, antigen-binding fragment, variant or derivative thereof to be administered in combination with at least one other immune modulating therapy include, but are not limited to, the severity of the disease, the history of the disease, the potential for metastases, and the age, height, weight, health, and physical condition of the individual undergoing therapy. Similarly, the amount of anti-SEMA4D antibody, or fragment, variant, or derivative thereof, in combination with at least one other immune modulating therapy to be administered will be dependent upon the mode of administration and whether the subject will undergo a single dose or multiple doses of this agent.

In some embodiments of any of the methods and uses described herein, the dose of the anti-SEMA4D antibody is about 20 mg/kg, or an equivalent thereof for antigen binding fragments, variants or derivatives thereof. Such equivalents can readily be calculated by those of skill in the art.

In some embodiments of any of the methods and uses described herein, the anti-SEMA4D antibody, or fragment, variant, or derivative thereof, is administered every three weeks.

In some embodiments of any of the methods and uses described herein, the anti-SEMA4D antibody, or fragment, variant, or derivative thereof, the combination treatment includes an immune checkpoint inhibitor that is administered simultaneously with or sequentially to (in either order) an anti-SEMA4D antibody, or fragment, variant, or derivative thereof. In general, both drugs are administered on the same day so that the subject receives both drugs on the same cycle, e.g., every three weeks. Alternatively, these drugs can be administered on separate cycles.

In select embodiments of any of the methods and uses described herein, the immune checkpoint inhibitor is an anti-PD-1 antibody, and preferably is pembrolizumab. In select embodiments of any of the methods and uses described herein, the dose of pembrolizumab is about 200 mg.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); and in Ausubel et al. (1989) Current Protocols in Molecular Biology (John Wiley and Sons, Baltimore, Md.).

General principles of antibody engineering are set forth in Borrebaeck, ed. (1995) Antibody Engineering (2nd ed.; Oxford Univ. Press). General principles of protein engineering are set forth in Rickwood et al., eds. (1995) Protein Engineering, A Practical Approach (IRL Press at Oxford Univ. Press, Oxford, Eng.). General principles of antibodies and antibody-hapten binding are set forth in: Nisonoff (1984) Molecular Immunology (2nd ed.; Sinauer Associates, Sunderland, Mass.); and Steward (1984) Antibodies, Their Structure and Function (Chapman and Hall, New York, N.Y.). Additionally, standard methods in immunology known in the art and not specifically described are generally followed as in Current Protocols in Immunology, John Wiley & Sons, New York; Stites et al., eds. (1994) Basic and Clinical Immunology (8th ed; Appleton & Lange, Norwalk, Conn.) and Mishell and Shiigi (eds) (1980) Selected Methods in Cellular Immunology (W.H. Freeman and Co., NY).

Standard reference works setting forth general principles of immunology include Current Protocols in Immunology, John Wiley & Sons, New York; Klein (1982) J., Immunology: The Science of Self-Nonself Discrimination (John Wiley & Sons, NY); Kennett et al., eds. (1980) Monoclonal Antibodies, Hybridoma: A New Dimension in Biological Analyses (Plenum Press, NY); Campbell (1984) “Monoclonal Antibody Technology” in Laboratory Techniques in Biochemistry and Molecular Biology, ed. Burden et al., (Elsevere, Amsterdam); Goldsby et al., eds. (2000) Kuby Immunnology (4th ed.; H. Freemand & Co.); Roitt et al. (2001) Immunology (6th ed.; London: Mosby); Abbas et al. (2005) Cellular and Molecular Immunology (5th ed.; Elsevier Health Sciences Division); Kontermann and Dubel (2001) Antibody Engineering (Springer Verlan); Sambrook and Russell (2001) Molecular Cloning: A Laboratory Manual (Cold Spring Harbor Press); Lewin (2003) Genes VIII (Prentice Hall 2003); Harlow and Lane (1988) Antibodies: A Laboratory Manual (Cold Spring Harbor Press); Dieffenbach and Dveksler (2003) PCR Primer (Cold Spring Harbor Press).

All publications, patents, and patent applications are herein incorporated by reference in their entirety to the same extent as if each individual publication, patent or patent application was specifically and individually indicated to be incorporated by reference in its entirety.

### EXAMPLES

The examples presented herein represent certain embodiments of the present disclosure. However, it is to be understood that these examples are for illustration purposes only and do not intend, nor should any be construed, to be wholly definitive as to conditions and scope of this disclosure. The examples were carried out using standard techniques, which are known and routine to those of skill in the art, except where otherwise described in detail.

### Example 1. Pepinemab and Pembrolizumab Treatment for R/M HNSCC

Experimental design & Phase 1b. KEYNOTE-B84 (KN-B84; NCT04815720) is a single-arm open-label study evaluating the safety, efficacy, and pharmacokinetics/pharmacodynamics of pepinemab in combination with pembrolizumab (pembro) as first-line treatment of recurrent or metastatic head and neck cancer (R/M HNSCC). The study includes a safety run-in (n=3) and dose-expansion phase (maximum n=62). Pepinemab, previously found to be well-tolerated in combination with other immune checkpoint inhibitors was evaluated at the dose of 20 mg/kg, in combination with 200 mg pembro, both administered i.v. every three weeks (Q3W). Tumor biopsies were obtained from all subjects and assessed for PD-L1 expression levels using immunohistochemical staining. A CPS score was determined for each subject.

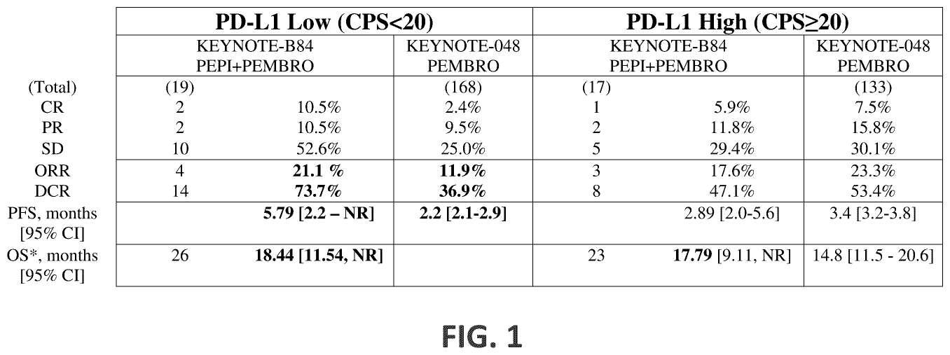

The primary efficacy endpoint is objective response rate (ORR), and the secondary endpoints are progression free survival (PFS), Duration of Response (DoR), and Overall Survival (OS), as well as exploratory biomarker analyses. Pre- and on-treatment biopsies were collected for evaluation of immune contexture in TME. Results: The safety run-in phase was completed (aka Phase 1), and the combination appeared to be well-tolerated with no dose limiting toxicities (DLTs) observed. Phase 2. An interim safety and efficacy analysis of the dose-expansion phase (maximum n=62) was conducted once 36 patients were enrolled and received their first scan at 9 weeks of treatment. Of this group, 19 subjects had PD-L1 levels with a CPS <20 (PD-L1 Low) and 17 subjects had PD-L1 levels with a CPS ≥20 (PD-L1 High). These KN-B84 results were compared with results from the KEYNOTE-048 study (KN-048), a Phase 3 study with single agent pembro (Burtness et al., J Clin Oncol. 40:2321-2332 (2022)), and the results are shown in the table of FIG. 1. Total represents the number of subjects in each arm of the study. The results show an approximately two-fold increase in in ORR, DCR, and PFS for the combination of pepinemab and pembrolizumab, as compared to the historical control of single agent pembrolizumab in the KEYNOTE-048 study. Among the CPS <20 population, ORR was 21.1% (2 CR and 2 PR), DCR was 73.7%, and median PFS was 5.79 months (KN-048 historical: ORR 11.9%, DCR 36.9%, PFS 2.2 months, n=168). DoR in all 4 responders in CPS <20 is ongoing except for one patient lost to COVID at 31 weeks post CR. One patient with tumor CPS <1 has an ongoing CR >17 months. In contrast, in the CPS ≥20 subgroup, the ORR was 17.6% (n=17), similar to historical data for single agent pembrolizumab.

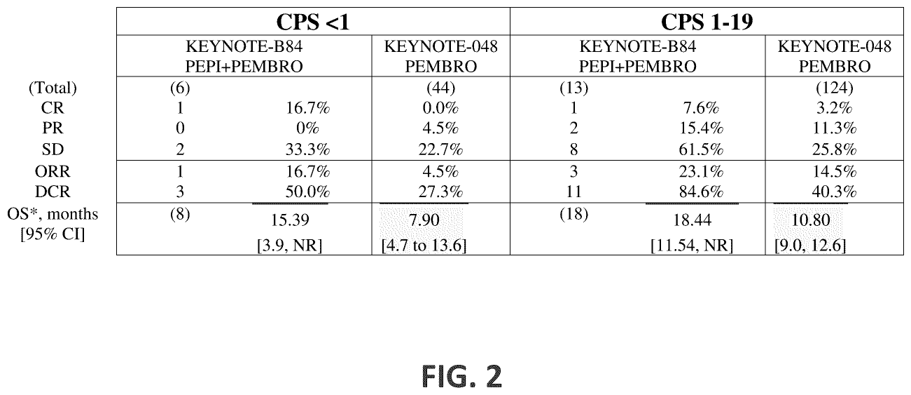

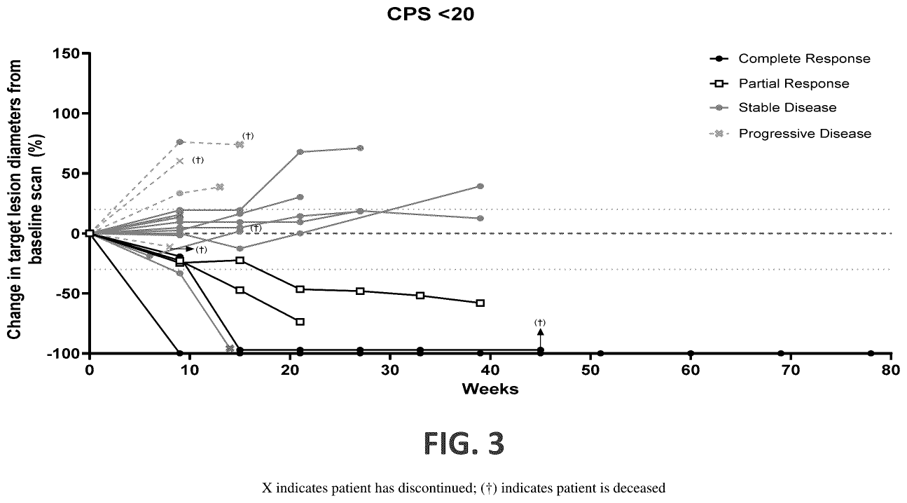

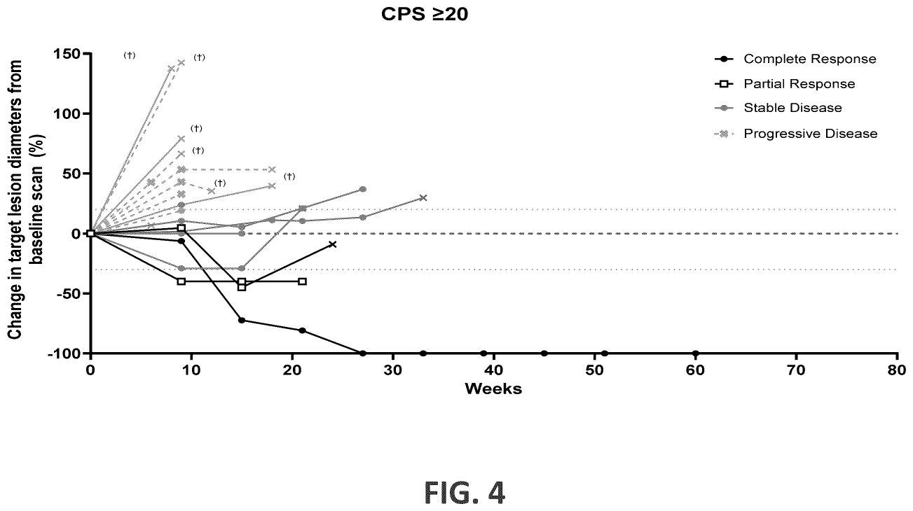

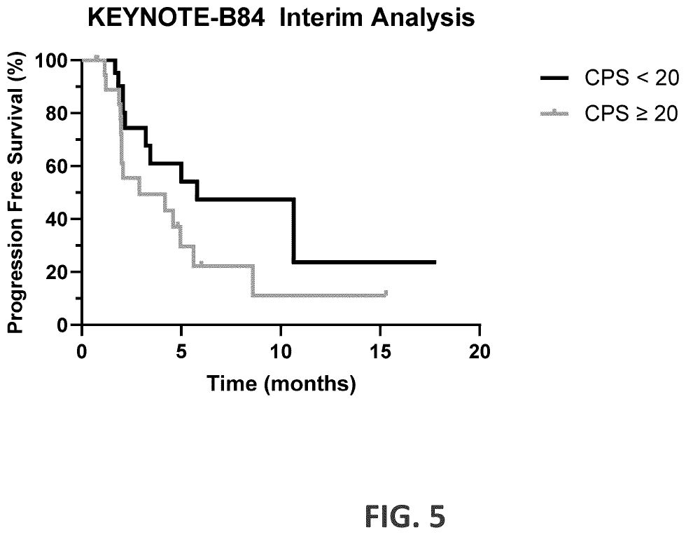

FIG. 2 provides a detailed breakdown of the foregoing PD-L1 CPS <20 results. The table in this figure shows CR, PR, SD for subjects with PD-L1 CPS <1, with PD-L1 ranging from CPS 1-19 and with PD-L1 CPS <20 on the combination therapy (KN-B84) compared to patients on pembrolizumab alone (KN-048). Total represents the number of subjects in each arm of the study. The changes in tumor size as a percent change from baseline are shown over the time course of the study for individual subjects for the CPS <20 cohort (FIG. 3) and for the subjects in the CPS ≥20 cohort (FIG. 4). FIG. 5 provides a plot of the probability of progression-free survival over time (in months) for subjects with PD-L1 of CPS <20 (black line) or a PD-L1 of CPS ≥20 (grey line).

Again, the results show an approximately two-fold increase in ORR, DCR and PFS in the CPS <20 cohort on the pepinemab plus pembro combination compared to the results in the KN-048 study.

These data in R/M HNSCC patients with PD-L1 Low tumors are consistent with a prior study demonstrating that pepinemab in combination with avelumab provided ˜2× increase in clinical benefit in patients with PD-L1-low NSCLC, as compared to historical control of single agent avelumab in the PD-L1 population (Shafique Clin Cancer Res 27:3630-3640, 2021)). However, in the NSCLC study, no distinction could be determined for responses between PD-L1 high and PD-L1 low tumors because the study did not enroll a sufficient number of patients with PD-L1 high tumors for comparison.

Many modifications and other embodiments of the embodiments set forth herein will come to mind to one skilled in the art to which this disclosure pertains, having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims and list of embodiments disclosed herein. Although specific terms are employed herein, they are used in a generic and descriptive sense only and not for purposes of limitation.

It should be understood the that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details can be made therein without departing from the spirit and scope of the disclosure as defined by the following claims. All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be herein incorporated by reference.

1. A method of selecting subjects having or suspected of having cancer for treatment with an isolated antibody or antigen-binding fragment thereof that specifically binds to semaphorin-4D (SEMA4D) and an effective amount of at least one other immune modulating therapy, preferably at least one immune checkpoint inhibitor, which comprises
(a) determining PD-L1 expression levels in tumor tissue or cancerous cells of a subject; and
(b) selecting the subject for treatment if
(i) the tumor tissue is identified as having a combined positive score (CPS) of <25 or a tumor proportion score (TPS) ranging from <50% to <80% for PD-L1 expression, or
(ii) the cancerous cells are identified as having a TPS ranging from <50% to <80% for PD-L1 expression.

2. A method for treating, inhibiting, delaying, or reducing malignant cell growth in a subject with cancer, comprising:
(a) determining PD-L1 expression levels in tumor tissue or cancerous cells of the subject; and
(b) administering to the subject an effective amount of an isolated antibody or antigen-binding fragment thereof that specifically binds to semaphorin-4D (SEMA4D) and an effective amount of at least one other immune modulating therapy, preferably at least one immune checkpoint inhibitor, if the tumor tissue or cells are identified as having either or both of a combined positive score (CPS) of <25 for PD-L1 expression or a tumor proportion score (TPS) ranging from <50% to <80%, thereby treating the subject.

3. The method of claim 1, wherein PD-L1 expression levels are determined by immunohistochemical analysis.

4. The method of claim 3 wherein said anti-SEMA4D antibody or antigen-binding fragment thereof inhibits SEMA4D interaction with its receptor.

5. The method of claim 4, wherein the receptor is Plexin-B1, Plexin-B2, CD72, or any combination thereof.

6. The method of claim 5, wherein the antibody or fragment thereof inhibits SEMA4D-mediated signal transduction.

7. The method of claim 4, wherein said anti-SEMA4D antibody or antigen-binding fragment thereof is selected from the group consisting of
(i) an antibody or antigen binding fragment thereof comprising a variable heavy chain (VH) region having VH CDRs 1-3 comprising amino acid sequences SEQ ID NOS:1, 2, and 3, respectively, and a variable light chain (VL) region comprising VL CDRs 1-3 comprising SEQ ID NOS:4, 5, and 6, respectively;
(ii) the antibody of (i), or antigen binding fragment thereof, wherein the VH and VL chains comprise, respectively, SEQ ID NO:7 and SEQ ID NO:8 (human), or SEQ ID NO:9 and SEQ ID NO:10 (mouse);
(iii) an antibody or antigen binding fragment thereof comprising a VH region having VH CDRs 1-3 comprising amino acid sequences SEQ ID NOS:11, 12, and 13, respectively; and a VL region comprising VL CDRs 1-3 comprising amino acid sequences SEQ ID NOS:14, 15, and 16, respectively; and
(iv) the antibody of (ii), or antigen binding fragment thereof, wherein the VH and VL chains comprise, respectively, SEQ ID NO:17 and SEQ ID NO:18.

8. The method of claim 1, wherein the at least one immune checkpoint inhibitor is an anti-CTLA4 antibody, an anti-PD-1 antibody, an anti-PD-L1 antibody, an anti-LAG3 antibody, an anti-TIGIT antibody, an anti-B7-H3 antibody or a combination thereof.

9. The method of claim 8, wherein the anti-PD-1 antibody is pembrolizumab, cemiplimab, dostarlimab or nivolumab, and preferably is pembrolizumab.

10. The method of claim 2, wherein the isolated antibody or antigen-binding fragment thereof and the at least one other immune modulating therapy, preferably the at least one immune checkpoint inhibitor, are administered separately or concurrently.

11. The method of claim 2, wherein administration of the combination of the isolated antibody or antigen-binding fragment thereof and the at least one other immune modulating therapy, preferably the at least one immune checkpoint inhibitor, results in enhanced therapeutic efficacy relative to administration of the at least one other immune modulating therapy, preferably the at least one immune checkpoint inhibitor, alone, wherein such enhancement is an approximately two-fold improvement in the objective response rate (ORR).

12. The method of claim 2, am which further comprise administering an immune modulating therapy, radiation or chemotherapy.

13. The method of claim 12, wherein the immune modulating therapy is selected from the group consisting of administration of a cancer vaccine, administration of an immunostimulatory agent, adoptive T cell or antibody therapy, administration of an immune checkpoint inhibitor, administration of a regulatory T cell (Treg) modulator, and a combination thereof.

14. The method of claim 13, wherein the immune modulating therapy comprises a second immune checkpoint inhibitor.

15. The method of claim 14, wherein the second immune checkpoint inhibitor is selected from the group consisting of an antibody or antigen-binding fragment thereof that specifically binds to CTLA4, PD-1, PD-L1, LAG3, TIM3, B7-H3, TIGIT or any combination thereof.

16. The method of claim 15, wherein the antibody or antigen-binding fragment of the second immune checkpoint inhibitor comprises the anti-PD-L1 antibody avelumab, atezolizumab, durvalumab.

17. The method of claim 13, wherein the immune modulating therapy comprises administration of a cancer vaccine.

18. The method of claim 13, wherein the Treg modulator is cyclophosphamide

19. The method of claim 1, wherein the cancer is carcinoma, lymphoma, blastoma, sarcoma, leukemia, squamous cell cancer, small-cell lung cancer, non-small cell lung cancer, adenocarcinoma of the lung, squamous carcinoma of the lung, cancer of the peritoneum, hepatocellular cancer, gastrointestinal cancer, gastric cancer, pancreatic cancer, neuroendocrine cancer, glioblastoma, cervical cancer, ovarian cancer, liver cancer, bladder cancer, brain cancer, hepatoma, breast cancer, colon cancer, colorectal cancer, endometrial or uterine carcinoma, esophageal cancer, salivary gland carcinoma, kidney cancer, liver cancer, prostate cancer, vulval cancer, thyroid cancer, head and neck cancer, or any combination thereof.

20. The method of claim 1, wherein the cancer is selected from the group consisting of locally advanced or recurrent or metastatic (in all of the following) head and neck cancer, non-small cell lung cancer, cervical cancer, urothelial carcinoma, esophogeal squamous cell carcinoma (ESCC), renal cell carcinoma, ovarian cancer, prostate cancer, bladder cancer, pancreatic cancer, gastrointestinal cancer, hepatocellular carcinoma, sarcoma, melanoma, triple negative breast cancer (TNBC), microsatellite instability/mismatch repair (MSI/MMR) deficient tumors and colorectal cancer.

21. A method of treating cancer in a human patient identified as having a tumor with low PD-L1 expression which comprises administering to the patient, as combination immunotherapy,
(a) an anti-semaphorin-4D (SEMA4D) antibody, or an antigen binding fragment thereof, and
(b) an immune checkpoint inhibitor,
wherein the tumor has a combined positive score (CPS) of <25 before the immune checkpoint inhibitor is administered.

22. The method of claim 21, wherein CPS is <20 or CPS is <10.

23. The method of claim 21, wherein said immune checkpoint inhibitor is an anti-PD-1 antibody, preferably pembrolizumab.

24. The method of claim 21, wherein said anti-SEMA4D antibody is pepinemab.

25. The method of claim 21, wherein said cancer is selected from the group consisting of locally advanced or recurrent or metastatic (in all of the following) head and neck cancer, non-small cell lung cancer, cervical cancer, urothelial carcinoma, esophogeal squamous cell carcinoma (ESCC), renal cell carcinoma, ovarian cancer, prostate cancer, bladder cancer, pancreatic cancer, gastrointestinal cancer, hepatocellular carcinoma, sarcoma, melanoma, triple negative breast cancer (TNBC), microsatellite instability/mismatch repair (MSI/MMR) deficient tumors and colorectal cancer.

26. A method of treating cancer in a human patient identified as having a tumor or cancerous cells with low PD-L1 expression which comprises administering to the patient, as combination immunotherapy,
(a) an anti-semaphorin-4D (SEMA4D) antibody, or an antigen binding fragment thereof, and
(b) an immune checkpoint inhibitor,
wherein the tumor has, or said cancerous cells have, a total proportion score (TPS) ranging from <50% to <80% before the immune checkpoint inhibitor is administered.

27. The method of claim 26, wherein TPS is <50.

28. The method of claim 26, wherein said immune checkpoint inhibitor is an anti-PD-1 antibody, preferably pembrolizumab.

29. The method of claim 26, wherein said anti-SEMA4D antibody is pepinemab.

30. The method of claim 26, wherein said cancer is selected from the group consisting of locally advanced or recurrent or metastatic (in all of the following) head and neck cancer, non-small cell lung cancer, cervical cancer, urothelial carcinoma, esophogeal squamous cell carcinoma (ESCC), renal cell carcinoma, ovarian cancer, prostate cancer, bladder cancer, pancreatic cancer, gastrointestinal cancer, hepatocellular carcinoma, sarcoma, melanoma, triple negative breast cancer (TNBC), microsatellite instability/mismatch repair (MSI/MMR) deficient tumors and colorectal cancer.

31. The method of claim 21, wherein said anti-SEMA4D antibody, or antigen binding fragment thereof, is administered at about 20 mg/kg.

32. The method of claim 21, wherein said anti-SEMA4D antibody, or antigen binding fragment thereof, is administered intravenously every three weeks.

33. The method of claim 21, wherein said immune checkpoint inhibitor is administered intravenously on the same cycle as said anti-SEMA4D antibody, or antigen binding fragment thereof.

34. The method of claim 21, wherein said anti-SEMA4D antibody, or antigen binding fragment thereof, and said immune checkpoint inhibitor are administered to the subject sequentially or simultaneously, optionally wherein said anti-SEMA4D antibody, or antigen binding fragment thereof, and said immune checkpoint inhibitor are contained in the same pharmaceutical composition or separate pharmaceutical compositions.

35. The method of claim 21, wherein said immune checkpoint inhibitor is an anti-PD-1 antibody.

36. The method of claim 35, wherein said anti-PD-1 antibody is pembrolizumab.

37. The method of claim 21, wherein said immune checkpoint inhibitor is pembrolizumab administered at a dosage of about 200 mg.


SEQUENCE LISTING (18 sequences)

SEQ ID NO: 1 - protein, 10 aa, synthetic construct
GYSFSDYYMH

SEQ ID NO: 2 - protein, 16 aa, synthetic construct
QINPTTGGASYNQKFK

SEQ ID NO: 3 - protein, 9 aa, synthetic construct
YYYGRHFDV

SEQ ID NO: 4 - protein, 15 aa, synthetic construct
KASQSVDYDGDSYMN

SEQ ID NO: 5 - protein, 7 aa, synthetic construct
AASNLES

SEQ ID NO: 6 - protein, 9 aa, synthetic construct
QQSNEDPYT

SEQ ID NO: 7 - protein, 118 aa, Homo sapiens
QVQLVQSGAEVKKPGSSVKVSCKASGYSFSDYYMHWVRQAPGQGLEWMGQINPTTGGASYNQKFKGKATITVDKSTSTAYMELSSLRSEDTAVYYCARYYYGRHFDVWGQGTTVTVSS

SEQ ID NO: 8 - protein, 111 aa, Homo sapiens
DIVMTQSPDSLAVSLGERATINCKASQSVDYDGDSYMNWYQQKPGQPPKLLIYAASNLESGVPDRFSGSGSGTDFTLTISSLQAEDVAVYYCQQSNEDPYTFGQGTKLEIK

SEQ ID NO: 9 - protein, 118 aa, Mus musculus
QVQLQQSGPELVKPGASVKISCKASGYSFSDYYMHWVKQSPENSLEWIGQINPTTGGASYNQKFKGKATLTVDKSSSTAYMQLKSLTSEESAVYYCTRYYYGRHFDVWGQGTTVTVSS

SEQ ID NO: 10 - protein, 111 aa, Mus musculus
DIVMTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIK

SEQ ID NO: 11 - protein, 5 aa, synthetic construct
DYWMV

SEQ ID NO: 12 - protein, 11 aa, synthetic construct
HMNQDGGARYY

SEQ ID NO: 13 - protein, 5 aa, synthetic construct
DPWGY

SEQ ID NO: 14 - protein, 11 aa, synthetic construct
SGDKLGDKYAV

SEQ ID NO: 15 - protein, 7 aa, synthetic construct
QDSKRPS

SEQ ID NO: 16 - protein, 10 aa, synthetic construct
QAWEQEAAWV

SEQ ID NO: 17 - protein, 113 aa, synthetic construct
EVQLVESGGGLVQPGGSLRLSCAASGFIFSDYWMVWVRQAPGKGLEYVAHMNQDGGARYYAESVRGRFTISRDNAKNSLYLQMNSLRAEDTAVYYCARDPWGYDSWGQGTLVT

SEQ ID NO: 18 - protein, 104 aa, synthetic construct
SYELTQPPSVSVSPGQTASITCSGDKLGDKYAVWYQQKPGQSPVLVIYQDSKRPSGIPERFSGSNSGNTATLTISGTQAMDEADYYCQAWEQEAAWVFGGGTKL